(19) United States
(12) Patent Application Publication
(10) Pub. No.: US 2026/0331293 A1
(43) Pub. Date: Oct. 22, 2026

(54) SYSTEMS AND METHODS FOR GENERATING A HEALTH OUTCOME PREDICTION

(71) Applicant: ResolveHD Inc., Halifax (CA)

(72) Inventors: Louis Alain Beaubien, Halifax (CA); Christopher George Crowell, Halifax (CA)

(21) Appl. No.: 19/637,599

(22) Filed: Apr. 2, 2026

Related U.S. Application Data
(63) Continuation of application No. PCT/CA2024/051330, filed on Oct. 7, 2024.
(60) Provisional application No. 63/588,530, filed on Oct. 6, 2023.

(51) Int. Cl. G16H 50/30; G16H 15/00

(52) U.S. Cl. CPC G16H 50/30; G16H 15/00

(57) ABSTRACT

Various systems and methods for generating a health outcome prediction for a patient are disclosed herein. The embodiments described involve operating a processor to receive a health condition of interest, receive a health dataset including patient data associated with the patient that includes demographic information about the patient, and clinical information about the patient. At least a portion of the health dataset is unstructured data and the health dataset originates from distinct data sources. The described embodiments then involve operating the processor to process the unstructured data to obtain structured data, extracting from the processed health dataset, features determined to be relevant to the health condition of interest, determine a risk score for the patient for the health condition of interest using a trained health prediction engine, based on the features extracted and generate and display a graphical output based on the risk score.

### CROSS-REFERENCE TO RELATED APPLICATION

The application is a continuation of International Application No. PCT/CA2024/051330, filed Oct. 7, 2024, which claims priority from U.S. Provisional Patent Application No. 63/588,530, filed on Oct. 6, 2023, the entire contents of which are hereby incorporated by reference for all purposes.

### FIELD

The present subject matter relates to systems for generating a health outcome prediction for a patient and methods of operating thereof.

### BACKGROUND

Traditionally, healthcare providers use their expertise and judgement to determine a patient's risk profile and predict the evolution of a patient's health condition. Based on their assessment, healthcare providers can then make decisions about the course of treatment, the monitoring plan or the therapy to be followed. However, healthcare providers typically only have access to limited information about their patients.

Electronic systems have been introduced to assist healthcare providers in evaluating their patients. These electronic systems typically employ electronic medical records (EMR) and generate a prediction about a patient's current or future health condition based on the information contained in the patient's EMR. However, information from electronic medical records can often be incomplete or can fail to fully capture characteristics that may affect a patient's health outcomes.

Further, data relevant to making health predictions often originates from disparate sources having different structures, and existing systems are typically only adapted for handling specific types of data structures or data from specific sources. In addition, these existing systems typically require a dataset having a fixed and predetermined composition and fail to recognize that information available for each patient can differ.

### SUMMARY OF VARIOUS EMBODIMENTS

In accordance with a first aspect of this disclosure, there is provided a system for generating a health outcome prediction for a patient. The system includes at least one processor configured to: receive a health condition of interest; receive a health dataset comprising at least patient data associated with the patient at least a portion of the health dataset being unstructured data, wherein the health dataset originates from a plurality of distinct data sources, and wherein the patient data comprises at least demographic information about the patient and clinical information; process the unstructured data of the health dataset to obtain structured data; extract from the processed health dataset, a plurality of features determined to be relevant to the health condition of interest; determine a risk score for the patient for the health condition of interest using a trained health prediction engine, based on the plurality of features extracted; and generate and display, on a graphical user interface, a graphical output based on the risk score.

In at least one embodiment, the health dataset includes one or more of genomic information, population data, lifestyle information, temporal information, environmental information, and sensor data.

In at least one embodiment, the at least one processor is configured to determine a recommendation based on the risk score and the health condition of interest.

In at least one embodiment, the recommendation is one of a treatment, a targeted therapy, a monitoring strategy.

In at least one embodiment, the recommendation is further based on environmental conditions and a type of health condition, and the recommendation is one of a resource allocation recommendation and an evacuation recommendation.

In at least one embodiment, the health dataset comprises real-time patient data from one of an emergency response dispatch system and emergency incident reports.

In at least one embodiment, the graphical output is a visual alert.

In at least one embodiment, the risk score is associated with a timeline.

In at least one embodiment, the health prediction engine determines the risk score according to a composition of the health dataset.

In at least one embodiment, the health prediction engine determines the risk score based on correlations between the plurality of relevant features and health outcomes.

In at least one embodiment, the unstructured data comprises data elements, and processing the unstructured data comprises: determining, via a parsing module, data element descriptors for each of the data elements of the unstructured data; identifying, via the parsing module, a corresponding parsed label for each of the data elements, the parsed label identified based on the data element descriptor; generating, via the parsing module, modified information data flows including the parsed labelled data elements; and analyzing, via a semantic context and analysis module, the modified information data flows to determine a meaning and context of the modified information data flows.

In at least one embodiment, identifying the corresponding parsed label comprises one of: matching a given data element according to a predefined parsed label mapping, and determining a given parsed label based on one or more of keywords and synonyms of the given data element.

In at least one embodiment, analyzing the modified information data flows comprises comparing the modified information data flows with analytic rules to identify one or more of errors and omissions in the modified information data flows.

In at least one embodiment, the parsing module is configured to determine one or more of a sensitivity level, a violation severity, a likelihood of occurrence, and a likelihood of detection for each data element.

In at least one embodiment, the semantic context and analysis module is further configured to generate a report including identified errors or omissions in the modified information data flows corresponding to the information data flows.

In at least one embodiment, determining the meaning and context of the modified information data flows comprises identifying patterns, sentiments and relationships within the modified information data flows.

In accordance with another aspect of this disclosure, there is provided a method for generating a health outcome prediction for a patient. The method involves: receiving a health condition of interest; receiving a health dataset comprising at least patient data associated with the patient, at least a portion of the health dataset being unstructured data, wherein the health dataset originates from a plurality of distinct data sources, and wherein the patient data comprises at least demographic information about the patient and clinical information; processing the unstructured data of the health dataset to obtain structured data; extracting from the processed health dataset, a plurality of features determined to be relevant to the health condition of interest; determining a risk score for the patient for the health condition of interest using a trained health prediction engine, based on the plurality of features extracted; and generating and displaying, on a graphical user interface, a graphical output based on the risk score.

In at least one embodiment, the health dataset includes one or more of genomic information, population data, lifestyle information, temporal information, environmental information, and sensor data.

In at least one embodiment, the method involves comprising determining a recommendation based on the risk score and the health condition of interest.

In at least one embodiment, the recommendation is one of a treatment, a targeted therapy, a monitoring strategy.

In at least one embodiment, the recommendation is further based on environmental conditions and a type of health condition, and the recommendation is one of a resource allocation recommendation and an evacuation recommendation.

In at least one embodiment, the risk score is associated with a timeline.

In at least one embodiment, the health prediction engine determines the risk score according to a composition of the health dataset.

In at least one embodiment, the health prediction engine determines the risk score based on correlations between the plurality of relevant features and health outcomes.

In at least one embodiment, the unstructured data comprises data elements, and processing the unstructured data comprises: determining, via a parsing module, data element descriptors for each of the data elements of the unstructured data; identifying, via the parsing module, a corresponding parsed label for each of the data elements, the parsed label identified based on the data element descriptor; generating, via the parsing module, modified information data flows including the parsed labelled data elements; and analyzing, via a semantic context and analysis module, the modified information data flows to determine a meaning and context of the modified information data flows.

In at least one embodiment, identifying the corresponding parsed label comprises one of: matching a given data element according to a predefined parsed label mapping, and determining a given parsed label based on one or more of keywords and synonyms of the given data element.

In at least one embodiment, analyzing the modified information data flows comprises comparing the modified information data flows with analytic rules to identify one or more of errors and omissions in the modified information data flows.

In at least one embodiment, the method involves determining, by the parsing module, one or more of a sensitivity level, a violation severity, a likelihood of occurrence, and a likelihood of detection for each data element.

In at least one embodiment, the method involves generating, by the semantic context and analysis module, a report including identified errors or omissions in the modified information data flows corresponding to the information data flows.

In at least one embodiment, the method involves determining the meaning and context of the modified information data flows comprises identifying patterns, sentiments and relationships within the modified information data flows.

It will be appreciated that the foregoing summary sets out representative aspects of embodiments to assist skilled readers in understanding the following detailed description. Other features and advantages of the present application will become apparent from the following detailed description taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 is a block diagram of an example embodiment of health outcome prediction system in communication with external components.

FIG. 2 is a flowchart of an example embodiment of a method for generating a health outcome prediction, in accordance with an example embodiment.

FIG. 3 is a block diagram showing modules of the system for managing and analyzing information data flows, in accordance with an example embodiment.

FIG. 4 is a screenshot of an example graphical user interface showing an output of the health outcome prediction system of FIG. 1.

FIG. 5 is a flow chart of an example embodiment of a method for managing and analyzing information data flows.

Further aspects and features of the example embodiments described herein will appear from the following description taken together with the accompanying drawings.

### DETAILED DESCRIPTION OF THE EMBODIMENTS

The headings and Abstract of the Disclosure provided herein are for convenience only and do not interpret the scope or meaning of the embodiments.

Various embodiments aligned with the principles outlined herein will be detailed below, offering examples of at least one embodiment of the claimed subject matter. It is important to note that no specific embodiment described in this document constrains or restricts any claimed subject matter. The claimed subject matter is not limited to devices, systems, or methods that incorporate all features of any individual embodiment discussed herein, nor is it confined to features that may be shared across multiple or all embodiments presented.

Additionally, it is possible that a device, system, or method mentioned in this document may not represent an embodiment of any claimed subject matter. Furthermore, any subject matter referenced herein that is not explicitly claimed in this document may be protected under a separate intellectual property filing, such as a continuing patent application. Any subject matter that is described herein that is not claimed in this document may be the subject matter of another protective instrument, for example, a continuing patent application, and the applicants, inventors or owners do not intend to abandon, disclaim or dedicate to the public any such subject matter by its disclosure in this document.

Furthermore, it should be noted that, for the sake of simplicity and clarity in illustration, reference numerals may be repeated across the figures to denote corresponding or analogous elements or steps. This approach helps to enhance understanding by providing continuity in the representation of similar concepts or processes throughout the illustrations.

Additionally, numerous specific details are included to ensure a comprehensive understanding of the embodiments presented herein, particularly in the context of healthcare applications. These details may encompass specific protocols, data flow mechanisms, or patient care strategies designed to illustrate the practical implementation of the described systems and methods. However, it is important to recognize that the embodiments described can be effectively practiced without relying on every specific detail outlined.

In some instances, well-established methods, procedures, and components that are commonly understood by those of ordinary skill in the art have not been elaborated upon in detail.

Moreover, the descriptions provided should not be construed as limiting the scope of the embodiments discussed. Rather, they serve to illustrate the innovative features and potential applications in a healthcare setting. The flexibility of these embodiments allows for adaptations and variations that can be tailored to specific healthcare needs, ensuring that they can be utilized effectively across different clinical scenarios and settings.

It is important to clarify that the terms “coupled” or “coupling,” as used throughout this document, can carry various meanings based on the specific context in which they are applied. These terms are versatile and may refer to different types of connections, including mechanical, electrical, or communicative relationships between elements.

For instance, in the context of healthcare technology, “coupled” may refer to two devices that are directly connected, such as a medical sensor and a monitoring system that work together to collect and analyze patient data in real time. Alternatively, these terms can also describe connections that occur through one or more intermediate elements or devices. For example, a wearable health monitoring device might be coupled to a smartphone app via Bluetooth, allowing for the transmission of data through an intermediary wireless signal.

The precise nature of the coupling—whether it involves electrical components, such as wires or circuits, or mechanical elements, like levers or connectors—will depend on the specific application and context being discussed.

Unless the context indicates otherwise, the terms “comprise,” along with its variations such as “comprises” and “comprising,” should be understood in an open and inclusive manner throughout the specification and the claims that follow. Specifically, these terms should be interpreted to mean “including, but not limited to.”

For example, when stating that a medical device “comprises” certain components, it implies that the device includes those specific components while also allowing for the possibility of additional features or functionalities that enhance its performance or applicability in clinical settings.

Throughout the present description, various terms should be interpreted as follows, unless the context specifies otherwise: singular articles and pronouns, such as “a,” “an,” and “he” or “she,” are intended to encompass their plural forms (e.g., “those,” “they”) and vice versa.

Moreover, gendered pronouns are to be understood to include their counterparts; for instance, “he” should also encompass “she,” and “his” should include “her” and should not be construed as limiting the described implementations, uses, or performances to any specific gender.

Additionally, further definitions for specific terms may be provided within this document. These definitions are intended to apply to both prior and subsequent occurrences of those terms, ensuring consistency and clarity throughout the text. This approach allows for a comprehensive understanding of the concepts being presented, facilitating clearer communication and interpretation of the subject matter related to healthcare practices, systems, and innovations.

It should also be noted that, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

For instance, if a healthcare protocol states that a patient should receive “medication A and/or medication B,” this means that the patient could be prescribed either medication A, medication B, or both medications simultaneously, depending on their clinical needs and treatment plan.

Additionally, the phrasing “X, Y, and/or Z” is intended to mean X, Y, Z, or any combination of these elements. For example, if a health system describes treatment options that include “physical therapy, occupational therapy, and/or speech therapy,” it indicates that patients may receive one type of therapy, multiple therapies, or a combination thereof, tailored to their specific rehabilitation needs.

It is important to understand that terms of degree, such as “substantially,” “about,” and “approximately,” as used throughout this document, refer to a reasonable margin of deviation from the modified term. These terms of degree may also be construed as including a deviation of the modified term, such as by 1%, 2%, 5% or 10%, for example, if this deviation does not negate the meaning of the term it modifies.

For instance, in clinical measurements, a medication dosage of “approximately 100 mg,” encompasses a slight deviation—such as 1%, 2%, 5%, or even 10%—that does not fundamentally alter the therapeutic effect or safety profile of the medication. Therefore, a dosage between 90 mg and 110 mg could still be considered effective and appropriate, as long as the variation does not compromise the efficacy or safety of the treatment.

It is important to note that when numerical ranges are referenced by their endpoints in this document, they encompass all numbers and fractions within that specified range. For instance, the range “1 to 5” includes not only the integers 1, 2, 3, 4,and 5, but also fractions such as 1.5, 2.75, and 3.90. This comprehensive interpretation ensures that all values within the range are considered.

Furthermore, it should be understood that every number mentioned, along with its fractional components, is presumed to be modified by the term “about.” This term indicates that there is an acceptable variation associated with the numerical value being referenced. The allowable deviation can be up to a specific percentage of the given number, ensuring that the overall meaning and implications of the number remain intact. For example, variations of 1%, 2%, 5%, or even 10% may be permissible, provided that these adjustments do not significantly alter the effectiveness or safety of a treatment, medication dosage, or clinical guideline.

Reference throughout this specification to “one embodiment”, “an embodiment”, “at least one embodiment” or “some embodiments” means that one or more particular features, structures, or characteristics may be combined in any suitable manner in one or more embodiments, unless otherwise specified to be not combinable or to be alternative options.

In a healthcare context a particular embodiment might include a combination of data analysis methods, patient monitoring technologies, and treatment protocols tailored to improve health outcomes for individuals with chronic conditions.

This adaptability allows for innovation and customization, which are essential in the rapidly changing landscape of healthcare. By permitting various combinations of features, the system can be designed to address specific clinical challenges, enhance patient engagement, and optimize treatment efficacy.

In this specification and the accompanying claims, the singular terms “a,” “an,” and “the” are intended to encompass plural references as well, unless the context explicitly indicates otherwise.

Additionally, it is important to clarify that the term “or” is generally utilized in its broadest sense, which is to imply “and/or” unless the context suggests a more restrictive interpretation. For example, in a healthcare setting, a statement like “a physician or a nurse” can be understood to include either role or both.

Throughout this specification and the appended claims, the term “communicative”—as used in phrases such as “communicative pathway,” “communicative coupling,” and variations like “communicatively coupled”—generally refers to any engineered system or arrangement designed for the transfer and exchange of information. This definition encompasses a broad range of mechanisms that facilitate communication between devices, systems, or components within healthcare technologies.

Examples of communicative pathways include, but are not limited to:

Electrically Conductive Pathways: These pathways consist of conductive materials, such as wires, that enable the transmission of electrical signals. In a healthcare context, this could refer to the wiring used in medical devices that monitor vital signs or connect different components of a medical system.

Physiological Signal Conduction: This refers to the natural pathways through which physiological signals, such as neural impulses or electrical signals in the heart, are transmitted within the human body. Technologies that interface with these biological systems, like electrocardiograms (ECGs) or electromyography (EMG) devices, leverage these natural pathways to gather critical health data.

Electromagnetically Radiative Pathways: These pathways facilitate communication via electromagnetic radiation, such as radio waves. This is commonly seen in wireless health monitoring devices that transmit patient data to healthcare providers without physical connections, enhancing mobility and convenience in patient care.

Examples of communicative couplings encompass various methods of establishing connections for information exchange, including:

Electrical Couplings: These involve direct connections between devices that allow for the flow of electrical signals, essential for devices like infusion pumps or diagnostic equipment.

Magnetic Couplings: This refers to connections established using magnetic fields to facilitate communication, which can be particularly useful in certain medical imaging technologies.

Radio Couplings: This type of coupling employs radio frequencies for communication, enabling devices such as wearable health monitors to send data to smartphones or cloud-based systems for analysis and monitoring.

Any Combination Thereof: The communicative pathways and couplings can be utilized in various combinations to optimize information transfer and enhance the functionality of healthcare technologies.

The term “communicative” encompasses a wide array of engineered solutions that facilitate information transfer in healthcare settings, supporting the development of integrated systems that improve patient monitoring, diagnosis, and overall healthcare delivery.

Throughout this specification and the appended claims, infinitive verb forms are frequently employed to convey actions and functionalities associated with the described systems and methods. Examples of these infinitive verbs include, but are not limited to, “to detect,” “to provide,” “to transmit,” “to communicate,” “to process,” and “to route.”

Unless the specific context dictates otherwise, these infinitive verb forms should be interpreted in an open and inclusive manner. For instance, “to detect” should be understood as “to, at least, detect,” indicating that the system may have the capability to perform this action alongside other related functions. Similarly, “to provide” can be read as “to, at least, provide,” signifying that the system is designed to deliver this function as part of a broader array of capabilities.

For example, the phrase “to transmit” in a healthcare context can mean the transmission of data and the ability to securely send patient information, alerts, or diagnostic results across various healthcare platforms and devices.

Some of the example embodiments of the systems, devices, or methods outlined in this specification can be realized through a combination of hardware and software components. For instance, certain embodiments may utilize one or more computer programs that execute on programmable devices, which include at least one processing element and at least one data storage element. These storage elements can consist of both volatile memory (such as RAM) and non-volatile memory (such as flash storage or hard drives).

In a healthcare context, these programmable devices might be employed in various applications, such as patient monitoring systems, electronic health records (EHR) platforms, or medical imaging devices. These devices typically feature at least one input device, including, but not limited to, a keyboard, mouse, touchscreen, or voice recognition system or any other input device that can allow healthcare professionals to interact with the system, input patient data, or navigate through applications.

Furthermore, the devices also incorporate at least one output device to convey information to users such as healthcare professionals. Examples of output devices include display screens for presenting patient data, printers for generating reports, and wireless radios for transmitting information to other devices or healthcare systems. The specific combination off input and output devices can vary depending on the application and requirements of the healthcare environment.

It should be noted that certain elements utilized in implementing at least part of the embodiments described herein may be developed using software written in high-level procedural languages, including object-oriented programming languages.

For example, the program code may be written in languages such as C™ or C++™, which are commonly used in healthcare applications due to their performance and control over system resources. These languages facilitate the creation of modular and class-based designs, as recognized by those of ordinary skill in object-oriented programming. This modular design allows different components of the software to be developed, tested, and integrated independently, promoting efficiency and scalability in healthcare technology solutions.

In addition to high-level languages, some elements of the embodiments described herein may also be implemented using lower-level programming methods, such as assembly language or machine language. These languages can provide the necessary performance and efficiency required for specific tasks, especially in resource-constrained environments, such as embedded systems used in medical devices. Moreover, firmware may be utilized where necessary, offering direct control over hardware components and ensuring reliable operation in critical applications.

The combination of high-level and low-level programming can enable the embodiments described herein to deliver robust, effective solutions that enhance patient care, streamline workflows, and improve overall healthcare delivery.

At least some of the software programs utilized to implement at least one of the embodiments discussed in this document may be stored on various types of storage media or devices that are readable by either general-purpose or specialized programmable devices.

Moreover, some of the programs associated with the systems and methods described in the embodiments herein may be distributed as part of a computer program product. This product consists of a computer-readable medium that contains computer-usable instructions, including program code designed for execution by one or more processors.

The program code can either be pre-installed and embedded during the manufacturing process of the device or later added as an update to enhance the functionality of an already deployed computing system. This flexibility allows healthcare organizations to keep their systems up-to-date with the latest features and improvements.

The computer-readable medium can take various forms, particularly non-transitory forms, which may include but are not limited to diskettes, compact disks (CDs), magnetic tapes, microchips, and other magnetic or electronic storage devices. These mediums ensure that the software can be reliably stored and accessed as needed within healthcare environments.

In addition to non-transitory mediums, at least one embodiment may utilize transitory mediums, including, but not limited to, wire-line transmissions, satellite communications, and internet-based transfers (e.g., downloads) and can allow healthcare providers to update their systems with new software or patches without the need for physical media.

Furthermore, the computer-usable instructions may be available in various formats, which can include both compiled and non-compiled code.

Accordingly, any module, unit, component, server, computer, terminal, or device described herein that executes software instructions may incorporate or have access to various forms of computer-readable media. This includes a range of storage solutions, such as removable and non-removable data storage devices, which can include magnetic disks, optical disks, or magnetic tape.

Healthcare providers typically rely on their expertise and judgement to assess patients'health outcomes and predict the evolution of health conditions and make decisions based on their assessments and predictions. Healthcare providers, however, typically only have access to limited information about their patients, for example, information included in a patient's health record.

Some electronic systems exist to assist healthcare providers in evaluating their patients. These electronic systems typically extract information from electronic medical records and generate a prediction or an assessment based on the information extracted and known risk factors for various health conditions. However, information from electronic medical records can often be incomplete, depending on the health services previously provided to patients, and collection strategies employed by healthcare providers, and can omit factors that can affect a patient's health outcome.

Some electronic systems can use other types of data, in addition to electronic medical records. However, data relevant to making health predictions can originate from heterogenous, disparate sources that can have different structures or be unstructured and different formats, and existing systems are typically only adapted for handling specific types of data structures or formats, or data from specific sources. In addition, these existing systems typically require a dataset having a fixed and predetermined makeup, and fail to recognize that information available for each patient can differ. Since these systems typically require a dataset having a predetermined makeup, an incomplete dataset can cause these systems to fail to make a health prediction.

Described herein are various example embodiments for generating a health outcome prediction. The described embodiments can generate a health outcome prediction using a dataset that can include clinical data and demographic data and that can additionally include genomic information, lifestyle information, temporal information, environmental information, and any other information that may be relevant to predicting a health outcome of the patient. However, the described embodiments do not require a dataset that includes all of these data types to generate a health outcome prediction. The dataset used by the described embodiment can be a heterogenous dataset including data from various sources and/or in various formats and can include unstructured data.

As described, in healthcare settings, data is often collected from various platforms, such as electronic health records (EHRs), laboratory information systems, medical imaging devices, and patient monitoring equipment. The described embodiments can standardize the data within the dataset, allowing for the integration and analysis of information data flows from diverse sources.

By establishing a common framework for data representation, the described embodiments can ensure that all incoming data, regardless of its origin, adheres to uniform standards so that the incoming data can be combined and analyzed effectively. For example, as will be described, the described embodiments can enable patient demographic data collected from a registration system to be integrated with clinical data obtained from diagnostic tests.

The standardization of data can enhance the ability to conduct meaningful analyses across multiple datasets and can allow healthcare providers to derive valuable insights that can inform clinical decision-making and improve patient care. At least one of the embodiments described herein can enable healthcare practitioners to identify trends in patient outcomes, monitor the effectiveness of treatment protocols, and optimize resource allocation, by integrating and analyzing standardized data from different departments within a healthcare facility.

The standardized approach used in the described embodiments can support compliance with regulatory requirements and facilitate data sharing among healthcare providers, researchers, and public health organizations. By using consistent data formats, organizations can improve collaboration and communication, ensuring that stakeholders can access and interpret shared information accurately.

The embodiments described herein can promote a more cohesive understanding of patient information, which can lead to enhanced healthcare delivery, better patient outcomes, and more efficient healthcare operations by ensuring that all relevant data is accessible and actionable in a standardized manner.

The described embodiments can generate a health outcome prediction by extracting relevant features from the health dataset and applying the relevant features to a health prediction engine.

At least one of the described embodiments can be integrated with real-time data sources and generate health outcome predictions in real-time or near real-time as the real-time data sources are updated. At least one of the described embodiments can ingest data flows without disrupting or interrupting the data flows and form health datasets from the data flows.

The described embodiments can be used by healthcare providers to predict the evolution of a patient's health condition and improve decision-making.

At least one of the embodiments described herein can be integrated into clinical decision support systems and/or electronic health records, enabling health providers to view health outcome predictions and in some cases, recommendations within existing workflows.

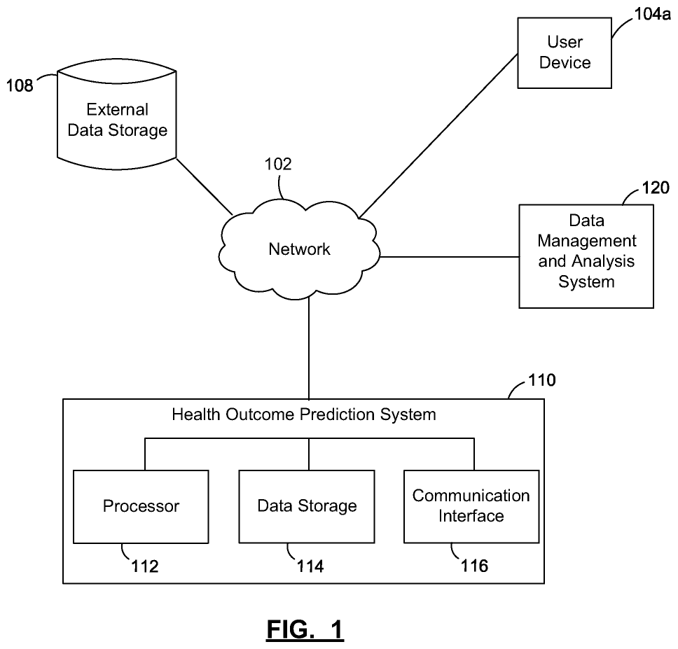

Referring first to FIG. 1, shown therein is a block diagram of an example embodiment of health outcome prediction system 110 in communication with a user device 104, an external data storage 108 and a data management analysis system 120 via a network 102. The health outcome prediction system 110 can communicate with the user device 104 over a local area or a wide geographic area via the network 102. The components 102, 104, 108, 110, 112, 114, 116 shown in the block diagram are provided as an example and there can be other embodiments with different components or a different configuration of components.

The health outcome prediction system 110 includes a computer server with a processor 112, a data storage 114 and a communication interface 116. The health outcome prediction system 110 can be implemented with more than one computer server distributed over a wide geographic area and connected via the network 102. The processor 112, the data storage 114 and the communication interface 116 may be combined into a fewer number of components or may be separated into further components depending on the scale of operation. The health outcome prediction system 110 can be configured to determine a risk score for a patient, for a health condition of interest and display an output corresponding to the risk score. The health outcome prediction system 110 can be configured to generate an alert based on the risk score and can be configured to determine a recommendation based on the risk score and the health condition of interest.

The processor 112 can be implemented with any suitable processor, controller, digital signal processor, graphics processing unit, application specific integrated circuits (ASICs), and/or field programmable gate arrays (FPGAs) that can provide sufficient processing power for the configuration, purposes and requirements of the health outcome prediction system 110. The processor 112 can include more than one processor with each processor being configured to perform different dedicated tasks.

The communication interface 116 can include any interface that enables the health outcome prediction system 110 to communicate with various devices and other systems. For example, the communication interface 116 can receive inputs (e.g., a health dataset, a health condition of interest) from the user device 104 and store the inputs in the data storage 114 or external data storage 108. The processor 112 can then process the inputs as described herein. The communication interface 116 can transmit outputs of the health outcome prediction system 110 (e.g., a risk score, an output based on the risk score) to the user device 104.

The communication interface 116 can include at least one of a serial port, a parallel port or a USB port, in some embodiments. The communication interface 116 may also include an interface to component via one or more of an Internet, Local Area Network (LAN), Ethernet, Firewire, modem, fiber, or digital subscriber line connection. Various combinations of these elements may be incorporated within the communication interface 116. For example, the communication interface 116 may receive input from various input devices, such as a mouse, a keyboard, a touch screen, a thumbwheel, a trackpad, a track-ball, a card-reader, voice recognition software and the like depending on the requirements and implementation of the health outcome prediction system 110.

The data storage 114 includes volatile and non-volatile memory components such as RAM, ROM, one or more hard drives, one or more flash drives or some other suitable data storage elements such as disk drives. The data storage 114 can include one or more databases that can store inputs, received by the health outcome prediction system 110 (e.g., a health condition of interest, a prediction timeline, health dataset) and/or outputs generated by the heath outcome prediction system 110 (e.g., risk scores, recommendations, graphical outputs). The data storage 114 can store training data for training the health prediction engine and/or the health prediction engine itself.

The external data storage 108 can store data similar to that of the data storage 114. The data stored in the external data storage 108 can be retrieved by the health outcome prediction system 110 via the network 102. The external data storage 108 can be cloud-based or a local data storage.

The user device 104 can include any electronic device capable of communicating with other devices through a network such as the network 102. A user device 104 can couple to the network 102 through a wired or wireless connection. Each user device 104 can include a processor and memory, and may be an electronic tablet device, a personal computer, workstation, server, portable computer, mobile device, personal digital assistant, laptop, smart phone, WAP phone, an interactive television, video display terminals, gaming consoles, and portable electronic devices or any combination of these. The user device 104 can be used for collecting data used as inputs for the health outcome prediction system 110 and/or for inputting a health condition of interest and/or for viewing an output of the health outcome prediction system 110. The user device 104 can a device conventionally used for collecting health data and may not be a device dedicated to collecting data for the health outcome prediction system 110.

The data management and analysis system 120 can be configured to process a health dataset used by the health outcome prediction system 110. The data management and analysis system 120 can be configured to process data from disparate data sources to generate a processed health dataset that can be used for generating a risk score. In some embodiments, the data management and analysis system 120 can be implemented by the health outcome prediction system 110. For example, the data management and analysis system 120 can be a subsystem of the health outcome prediction system 110.

The network 102 can include any network capable of carrying data, including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signaling network, fixed line, local area network, wide area network, and others, including any combination of these, capable of interfacing with, and enabling communication between, the data management and analysis system 110, the external data storage 108, and the user devices 104.

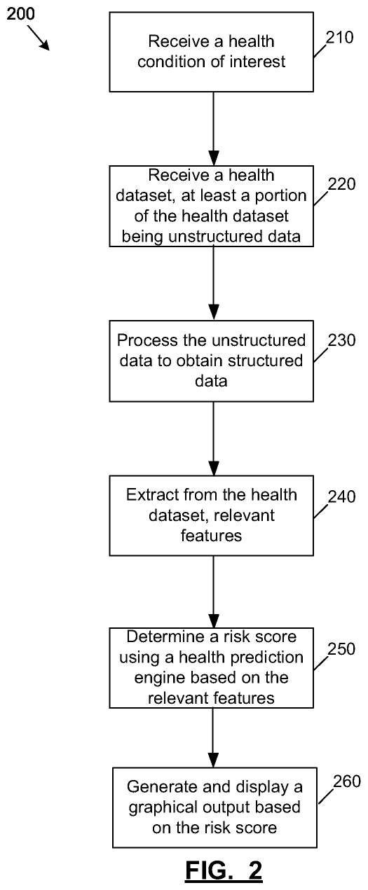

Referring to FIG. 2, shown therein is a flowchart of a method 200 of generating a health outcome prediction for a patient, according to an embodiment. The method 200 can be implemented by the health outcome prediction system 110.

At step 210, the health outcome prediction system 110 receives an input indicating one or more health conditions of interest for which a health outcome prediction is to be generated. The type of input can vary, depending on the implementation of the health outcome prediction system 110.

The health condition of interest(s) can be provided by a user, such as a healthcare provider, through user device 104. For example, the healthcare provider may select specific health conditions from a dropdown menu or enter them manually, indicating that they wish to assess the patient's risk for health conditions like diabetes or obesity, based on the healthcare provider's clinical assessment of the health conditions that may be experienced by the patient.

Alternatively, in at least one embodiment, the health outcome prediction system 110 can automatically identify the health condition(s) of interest based on data received. For example, if the health outcome prediction system 110 has access to a comprehensive dataset that includes patient history, symptoms, lab results, and other relevant health information, the health outcome prediction system 110 can analyze this data to identify health conditions of interest. For instance, if a patient's records indicate high blood glucose levels and a history of hypertension, the health outcome prediction system 110 may automatically recognize that diabetes is a health condition of interest.

Alternatively, in at least one embodiment, the health condition(s) of interest can include a combination of both user inputted health condition(s) and automatically detected health condition(s). For example, a healthcare provider may indicate a specific health condition of interest, and the health outcome prediction system 110 can suggest additional health conditions of interest based on the patient's overall health profile and risk factors identified in the data.

At 220, the health outcome prediction system 110 receives a health dataset. The health dataset includes patient data associated with and specific to the patient. The patient data includes at least demographic information about the patient (e.g., age, gender, race) and clinical information about the patient (e.g., health conditions experienced by the patient, comorbidities, therapy history, clinical presentation). T At least a portion of the health dataset is unstructured data (i.e., data having no predefined format). For example, the health dataset can include free text notes, such as clinical notes or laboratory reports and images such as medical images.

The data included in the health dataset can originate from a plurality of distinct data sources. For example, the health dataset can include data received from various user devices 104. As another example, the health dataset can include data retrieved from a data storage that stores data originating from different sources, for example, external data storage 108. As a further example, the health dataset can include data received from a combination of one or more user devices 104 and data retrieved from a data storage such as external data storage 108. The data sources and the data originating from the data sources can be of different types and/or be presented in different formats.

The health dataset can include data in any format, including text, audio and images.

In some embodiments, the health dataset includes one or more of genomic information, lifestyle information, temporal information and/or sensor data captured by wearable device(s) worn by the patient. Temporal information can include timing information associated with the health condition of interest. For example, temporal information can include the timing since diagnosis of the health condition, a symptom onset, timing information associated with administering a treatment. Genomic information can include information about gene mutations and expression levels of biomarkers strongly correlated with therapy response.

When compared to conventional systems, the health outcome prediction system 110 can use more diverse data to generate the health outcome prediction.

As explained, the health dataset can include data originating from a plurality of sources. For example, the health dataset can include data obtained from wearable health devices such as smartwatches, fitness trackers, or wearable sensors that monitor patient activity levels, heart rate, blood pressure, and other real-time physiological data.

As another example, the health dataset can include data obtained from smartphones, tablets and other mobile devices used by patients or healthcare providers to input health information, symptoms, or track medication adherence.

As another example, the health dataset can include data obtained from home monitoring devices such as glucometers, thermometers, and pulse oximeters that collect vital signs or diagnostic data from patients in their home environment.

As explained, the health dataset can include data retrieved from data storages, such as the external data storage 108. The data storages can be cloud-based or local, and can include data from electronic health records that include patient medical histories, diagnoses, medications, allergies, and lab results; data from laboratory information systems (LIS), including blood tests, biopsies, imaging results, and genetic testing reports; data from medical imaging databases, including X-rays, magnetic resonance imaging (MRI), computed tomography (CT) scans, and other radiology images used to support diagnosis; data from pharmacy databases, including prescription records, medication history, and pharmacological interventions; data from insurance claims systems, including information regarding patient treatments, services received, and healthcare utilization patterns; data from public health databases, including population health data, disease registries, and demographic trends that provide context for patient-level data; data from government health databases, including population health statistics, epidemiological data, or disease surveillance systems that track outbreaks and trends; data from clinical research databases, including information from clinical trials, observational studies, and published research relevant to patient conditions and treatments; and/or data from genomic databases, including genetic and genomic information that can aid in personalized medicine, identifying risk factors or predicting disease progression.

The use of various data sources can provide the the health outcome prediction system 110 a more accurate and comprehensive understanding of a patient's health.

At 230, the health outcome prediction system 110 preprocesses the unstructured data included in the health dataset to obtain structured data.

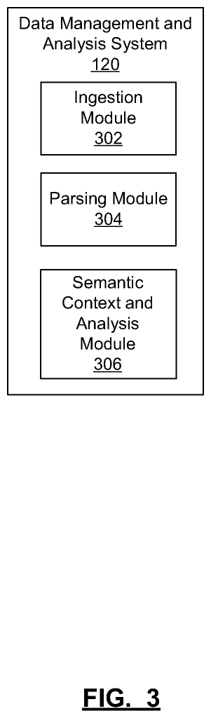

In at least some embodiments, the health outcome prediction system 110 preprocesses the health dataset to obtain a processed health dataset. The health dataset, including the unstructured data can be preprocessed using the data management and analysis system 120, described in more detail with reference to FIG. 3.

The unstructured data can be preprocessed to extract features representative of the meaning and context of the unstructured data. For example, in embodiments where the health dataset includes unstructured clinical notes, the health outcome prediction system 110 can preprocess the clinical notes to identify a diagnosis of the patient, medications taken by the patient, symptoms experienced by the and/or treatment plans undergone by the patient. For example, the health outcome prediction system 110 may extract from unstructured clinical notes the phrase “patient diagnosed with diabetes” and the diagnosis of diabetes. In embodiments where the health dataset includes temporal information, the temporal information can be processed to extract relevant timing information. For example, the health outcome prediction system 110 can extract timing details associated with diagnosis of the patient and/or the duration of the treatment of the patient.

The features extracted can vary, depending on the implementation of the health outcome prediction system 110.

In at least one embodiment, the unstructured data can be preprocessed to extract features using conventional techniques for feature extraction. For example, feature extraction techniques can include Natural Language Processing (NLP) algorithms, machine learning, or domain-specific rules. These techniques analyze the data to identify recurring patterns, key terms, and relevant contexts that are known to have predictive value in healthcare. The health outcome prediction system 110. may also use existing knowledge bases or clinical guidelines to extract features. For example, certain diagnoses or medications may be extracted from the health dataset due to their known impact on patient health.

In at least one embodiment, the health dataset can be preprocessed to clean the data included the health dataset to correct missing information or incomplete information. The health dataset can be preprocessed to normalize and/or scale the data. For example, numerical values may be scaled and/or standardized and textual data can be processed to identify terms with similar meanings.

In at least one embodiment, each data type can be preprocessed by a separate module and the modules can be executed in parallel, allowing the health dataset to be preprocessed more efficiently. For example, the health outcome prediction system 110 can include a module for preprocessing genomic information, a module for preprocessing demographic information, a module for preprocessing clinical information, etc.

At step 240, the health outcome prediction system 110 extracts features from the processed health dataset that have been determined to be relevant to the health condition of interest. The features determined to be relevant can be identified during a training process of a health prediction engine. During training of the health prediction engine, the health outcome prediction system 110 can analyze historical data related to various health conditions and learns which features—such as symptoms, diagnostic results, medications, and lifestyle factors—are most predictive of certain outcomes, such as the development of diabetes or the likelihood of recovery from a particular treatment.

The features can be determined via dimensionality reduction using techniques such as Principal Component Analysis (PCA), Feature Selection, or L1 Regularization (Lasso) to reduce the feature dataset's complexity. For example, in a dataset containing a large number of potentially relevant features, only those that have a strong correlation with the condition of interest—such as high blood pressure or elevated cholesterol for predicting heart disease—may be retained.

For example, the health prediction engine can identify features relevant to type 2 diabetes by analyzing historical patient data and identifying correlations. For example, identified relevant features can include as body mass index (BMI), family history of diabetes, and fasting blood glucose levels. During dimensionality reduction, features that do not include significantly influence diabetes risk—such as unrelated lab tests or irrelevant demographic information—may not be retained.

By extracting relevant features, the health outcome prediction system 110 can apply a prediction engine to only those features that have been determined to be relevant, rather than the entire dataset, reducing the processing resources required for generating a health outcome prediction system 110 and allowing a health outcome prediction to be generated in a more efficient manner.

During the training process, the health prediction engine can be trained to identify features of a health dataset that are most strongly correlated with particular health outcomes and with particular health conditions. For example, during the training process, the health prediction engine can be trained to identify which genomic markers are relevant to a particular health condition by identifying correlations between genomic markers and health conditions. The health prediction engine can be trained using one or more of feature selection techniques, recursive feature elimination and deep learning-based methods. The health prediction engine can be trained to identify features within a health dataset that are most relevant to a health prediction outcome and determine the risk score based on the identified features present in the health dataset received at 220.

The features extracted can vary depending on the composition of the health dataset and can vary on the type of data included in the health dataset. For example, the health outcome prediction system 110 can determine that a given health dataset includes demographic information, population data and clinical data but does not include genomic data, temporal information, sensor data, or lifestyle information and only extract relevant features associated with demographic information, population data and clinical data.

In this example, example extracted features may include age, gender, and ethnicity from the demographic data (Feature A, Feature B, Feature C), disease prevalence rates or geographic distribution from the population data (Feature D, Feature E), and diagnosis codes, lab results, or treatment history from the clinical data (Feature F, Feature G, Feature H).

The health outcome prediction system 110 can dynamically tailor the relevant features extraction process to the available data types, avoiding unnecessary processing data that may not be included in the health dataset.

At 250, the health outcome prediction system 110 determines a risk score for the patient for the health condition of interest using the health prediction engine. The health prediction engine can be trained to generate risks scores using the extracted features. The health prediction engine can include a plurality of models trained to determine a risk score, according to the type of data. The risk score can be an aggregate assessment of the patient's overall risk for the health condition of interest.

The health prediction engine can be trained using public datasets (e.g., public health data including population-level data and anonymized data about individuals) and/or historical health datasets for which a health outcome is known. The health prediction engine can be an engine that is trained to identify relationships between data elements (e.g., features) in a health dataset and health outcomes. For example, the health prediction engine can be an engine that is trained to identify the likelihood of a clinical condition leading to a particular health outcome and that is trained to identify the likelihood of a particular health outcome based on multiple data types (e.g., genomic features extracted from genomic information, clinical features extracted from clinical information, temporal features extracted from temporal information). For example, some combination of features corresponding to factors may be correlated with particular health conditions being exacerbated.

The health prediction engine can include one or more of classification models (e.g., logistic regression models, random forest models, support vector machines), probabilistic models (e.g., neural networks, Bayesian models) and time series model (e.g., Cox proportional hazard models). The health prediction engine can include models specific to the demographic information of the patient so that the risk score generated for the patient can be specific to the patient's demographic profile, for example, specific to the age, gender, ethnicity, and/or other demographic factors of the patient

In at least one embodiment, the health prediction engine can select the models to be applied to the extracted features based on the extracted features and in some cases, the composition of the health dataset (i.e., the types of data included in the health dataset).

For example, based on the determination that a given heath dataset does not include genomic information, the health prediction engine can apply one or more models configured to generate a risk score without genomic information. As explained, the health prediction engine can include a plurality of trained models. Upon extracting the relevant features and in some cases, determining the composition of the health dataset, the health prediction engine can select the appropriate models for determining the risk score.

In at least one embodiment, the health prediction engine includes a plurality of models, each specifically trained to analyze and generate risk scores according to different types of data, such as health records, lifestyle data, demographic information, and clinical data. For example, the health prediction engine can include distinct models dedicated to processing electronic health records (EHRs), lifestyle data, and population-level information. Each of the models can evaluate the extracted features from each respective datatype.

For example, if the health dataset includes both clinical and lifestyle data, the health prediction engine may utilize a clinical data model to analyze clinical factors like diagnoses and treatment history, while a separate lifestyle data model may evaluate information related to exercise, diet, smoking habits and other lifestyle information.

The health prediction engine can generate a combined risk score based on the results of the individual models. For example, the health prediction engine can aggregate the results from each model and apply a weighted or ensemble approach to determine an overall risk score. By using multiple models, the health outcome prediction system 110 can consider a combination of factors from diverse datasets to provide an accurate and comprehensive risk score, as it

The prediction engine can be configured to select the appropriate classification models to apply to the relevant features, based on the data type(s), or can be pre-configured to identify which models to apply to specific types of data. For example, if the dataset contains clinical records and temporal information, the prediction engine may automatically apply a random forest model for classification tasks, along with a Cox proportional hazards model to handle time-based predictions related to disease progression or treatment outcomes. As another example, the prediction engine may automatically apply logistic regression models to demographic information.

As another example, if lifestyle information is included in the health dataset, the health prediction engine may probabilistic models such as neural networks to account for complex, non-linear relationships between lifestyle factors and health outcomes. Conversely, for clinical information, the prediction engine can prioritize classification models to generate risk predictions based on diagnoses and medical history.

By training the health prediction engine to generate risks scores according to the composition of the health dataset, the health prediction engine can generate risk scores even when a complete health dataset is not available.

In some embodiments, the risk score is associated with a timeline. For example, the risk score can indicate a risk over a period of time (e.g., 6 months, 1 year, etc.). As another example, the risk score can be provided over multiple periods of time (e.g., risk score over time period Δt₁, risk score over time period Δt₂). By associating the risk score with a time period, the health outcome prediction system 110 can predict the evolution of the health condition of interest, which can assist in the patient's healthcare provider's decision making.

In some embodiments, the timeline associated with the risk score is inputted by a user. For example, the user can specify that a risk score over a period of 6 months is desired. Alternatively, the timeline can vary, depending on the health condition of interest and/or on the implementation of the health outcome prediction system 110.

The risk score can provide valuable insights for healthcare providers to assess the likelihood of chronic conditions, such as heart disease, diabetes, or cancer, and to develop tailored treatment or preventive strategies. By associated the risk score with a timeline, the health outcome prediction system 110 can enable long-term patient monitoring, help guide ongoing treatment decisions and optimize clinical resource allocation and facilitate early intervention.

At 260, the health outcome prediction system 110 generates and displays a graphical output based on the risk score. The graphical output can include one or more of heatmaps, timelines, survival curves, probability distributions, etc. The health outcome prediction system 110 can additionally display the risk score. The risk score can be displayed as a numerical value (e.g., percentage, probability) or as a qualitative value (e.g., “high risk”, “moderate risk”, “low risk”) reflecting the likelihood that the patient will experience the health condition, such as developing type 2 diabetes or suffering a cardiac event within a given time frame, if applicable.

In at least one embodiment, the health outcome prediction system 110 can identify and display factors associated with features contributing to the risk score. Displaying the factors can improve the transparency of the health prediction engine.

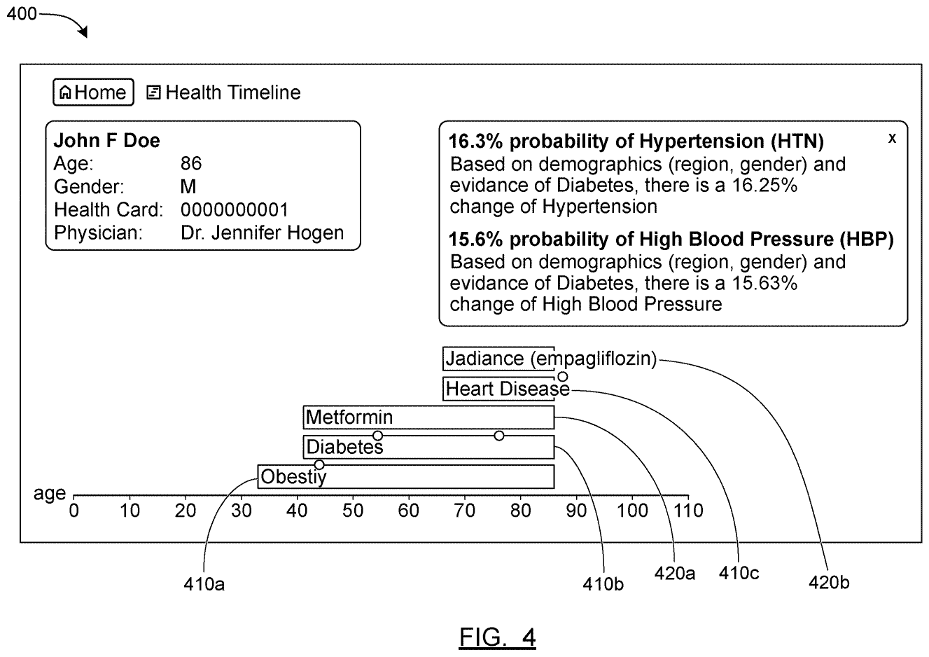

Referring briefly to FIG. 4, for example, which shows a screenshot of an example graphical user interface 400, the risk score can be presented as a percentage and a timeline associated with the risk score can be generated.

In the screenshot of FIG. 4, the health conditions of interest 410 are shown, along with associated diagnostic and therapeutic interventions 420 along a linear timeline. In the example shown, the patient is diagnosed with obesity during the patient's 30s and is predicted to continue to experience obesity until a later stage in life (i.e., the 80s) In at least one embodiment, key interventions, such as dietary recommendations, exercise programs, or weight management therapies that may be implemented throughout the patient's life to address this health condition may be displayed (not shown). In the example shown, the patient has been diagnosed with diabetes and may require ongoing management strategies. In at least one embodiment, specific diagnostic evaluations, such as blood sugar monitoring or HbA1c tests, as well as therapeutic interventions, such as the initiation of medications like metformin, to help manage their condition effectively may be shown. In the example shown, the initiation of metformin 420b is shown.

In some embodiments, the health outcome prediction system 110 generates an alert based on the risk score. For example, if the risk score exceeds a predetermined threshold, the health outcome prediction system 110 can be configured to output an alert. The alert can be a visual alert, that can be displayed on a graphical user interface.

In some embodiments, the health outcome prediction system 110 determines a recommendation based on the risk score and the health condition of interest. For example, the recommendation can be a course of action to decrease or manage the risk score, a treatment option, a targeted therapy and/or a monitoring strategy. The recommendations can be based on known recommendations for managing the health condition of interest.

In some embodiments, the risk score can be automatically updated when the health dataset is updated. For example, when additional data is introduced to the health dataset, the health outcome prediction system 110 can automatically determine an updated risk score.

As explained, the health outcome prediction system 110 can be used for supporting decision making. For example, the health outcome prediction system 110 can be used as part of an emergency medical services system.

In an emergency medical services scenario, the health outcome prediction system 110 can receive real-time audio transmissions. For example, the health outcome prediction system 110 can ingest audio transmissions from emergency medical technician (EMT) dispatches and/or radio channels. The audio transmissions can include patient data, for example the medical condition of the patient as reported by first responders (e.g., injuries, medical event) and additional information relevant to health outcome of the patient (e.g., environmental hazards that could impact patient health or first responder safety, temporal information related to incident reports and the evolution of the health or environmental situation). The health outcome prediction system 110 can additionally receive location information of the mobile units associated with the audio transmissions.

The health outcome prediction system 110 then processes the audio transmissions. For example, the health outcome prediction system 110 can process the audio transmissions using the data management and analysis system 120. The health outcome prediction system 110 can process the unstructured audio transmissions to obtain structured data. For example, the health outcome prediction system 110 can employ speech-to text algorithms to convert the audio transmissions into text and process the text or the audio transmissions to correct incomplete or unclear audio.

The health outcome prediction system 110 can also process the audio transmissions and/or text to normalize the contents of the audio transmissions. As explained, the data management and analysis system 120 can parse the data to map data elements. The data management and analysis system 120 can determine a meaning of the audio transmissions, so that terms having a similar meaning can be treated similarly.

The health outcome prediction system 110 can then extract features from the structured data that have been identified as being highly relevant to the assessment of a health condition. The features can be features identified during a training process of the health prediction engine. In a medical emergency medical services scenario, the health outcome prediction system 110 can extract keywords and phrases that characterize an injury type or a medical event experienced by the patient and that characterize the environment of the patient and can prioritize keywords that characterize a severe injury, a severe medical event, or hazardous environments.

The health outcome prediction system 110 then determines a risk score for the patient based on the extracted features. The health prediction engine can use the extracted features to determine a risk score for the patient. The health prediction engine can be trained using training data, including historical incident reports, audio data and/or first responder feedback and can be trained to determine a risk score overtime. The risk score can indicate a severity of the injury or medical event and a predicted patient outcome.

The health outcome prediction system 110 can generate a recommendation and/or an alert based on the risk score. For example, the health outcome prediction system 110 can determine that an injury is severe and requires immediate transportation to a trauma center, and can generate a visual alert on a graphical user interface.

The health outcome prediction system 110 can generate a recommendation based on the audio transmissions received. For example, the health outcome prediction system 110 can determine that the emergency is an environmental emergency due to a fire and generate a recommendation to evacuate the area. The recommendation can include a resource recommendation. For example, the health outcome prediction system 110 can recommend that additional ambulances be deployed to an area, or can recommend that patients be rerouted to specific hospitals based on the severity of their injury or the type of medical event.

The health outcome prediction system 110 can generate a report summarizing the recommendation, predicted needs based on the risk score and the type of emergency, as determined by the audio transmissions. The report can be transmitted to individuals involved in the provision of emergency medical services. For example, the report can be transmitted to emergency medical technicians, dispatchers and hospital personnel.

The health outcome prediction system 110 can generate and display a graphical output based on the risk score. For example, the health outcome prediction system 110 can cause incident maps showing first responder locations, injury severity and environmental risks to be displayed. The health outcome prediction system 110 can additionally display the recommendation(s).

Referring to FIG. 3, the diagram illustrates example modules of the data management and analysis system 120, which, as explained, can be configured to process data within the health dataset. In at least one embodiment, the data management and analysis system 120is part of the health outcome prediction system 110. The data management and analysis system 120 includes: an ingestion module 302, a parsing module 304, and a semantic context and analysis module 306. Each of these modules can be executed by a processor, such as processor 112, and may either run on a single processor or be distributed across multiple processors for scalability. The ingestion module 302, the parsing module 304, and the semantic context and analysis module 306 can be in communication with one another to pass data between various instances of one another.

The ingestion module 302 can be configured to retrieve and load data from various sources. This data can be passed to the parsing module 304, which can be configured to process and structure the data for analysis, for example, to transform the data included in the health dataset into a usable format. The semantic context and analysis module 306 can be configured to interpret the parsed data by adding context, extracting meaning, and applying analytics to derive insights relevant to health outcomes.

These modules can be configured to be in communication with one another, The modular configuration of the data management and analysis system 120 can enhance its flexibility and allow each component to be updated or scaled independently, improving both performance and resource allocation of the data management and analysis system 120.

The ingestion module 302 can be configured to efficiently handle a variety of information data flows, including data streams originating from various software application modules, such as a master data management application, which oversees and regulates the flow of information to and from data storage systems.

Additionally, the ingestion module 302 can manage data flows retrieved from these data stores and facilitate the transmission of this information to different software application modules. Furthermore, the ingestion module 302 can oversee data exchanges between software application modules and external systems, ensuring smooth and reliable communication.

The types of data flows processed by the ingestion module 302 can encompass a wide range of health data, including, but not limited to patient records, treatment histories, and genomic information. By effectively managing these diverse data streams, the ingestion module 302 can help ensure that all relevant health data is readily available for subsequent analysis and processing, including for generating a risk score for a patient.

The ingestion module 302 can be configured to process a variety of unstructured data types, such as text documents, CSV files, databases, web APIs, streamlined data sources, audio recordings, and sensor data. In the context of medical applications, the information data flows may include population-level health data sourced from government databases, patient demographics, and clinical records, etc. Additionally, the ingestion module 302 can ingest various other types that can be used for generating a health outcome prediction including: genomic data (i.e., information related to gene sequences, mutations, and expression profiles); lifestyle data: reflecting individual lifestyle factors such as diet, physical activity, and behavioral habits; clinical information including patient records detailing diagnoses, treatment plans, and medical histories; environmental data including Information regarding environmental factors that could impact health, such as air quality or geographical location; wearable device data including real-time data collected from fitness trackers and other health-monitoring devices that record metrics like heart rate, sleep patterns, and activity levels; and/or healthcare utilization data including information on healthcare services accessed by patients, including hospital visits, medications, and follow-up treatments.

Ingesting these diverse information data flows can involve both recording and monitoring these data streams to ensure a comprehensive collection of health-related information, facilitating accurate analysis and prediction within the system.

In at least one embodiment, the process of ingesting information data flows involves extracting data from multiple sources. The extraction method may differ based on the origin of the information data flows and the nature of the data contained within them. For instance, when dealing with text documents, the ingestion module 302 may use libraries specifically designed for various document formats, such as PDF or Word. In the case of databases, the ingestion module 302 can be configured to establish connections to these databases and execute queries to obtain the relevant data. Additionally, for APIs, the ingestion module 302 can be capable of sending requests to API endpoints, including the necessary authentication parameters to successfully retrieve the required information.

The information data flows may contain various data elements, which refer to the records or information included within those flows. For instance, in medical applications, these data flows can feature data elements that correspond to health record information, detailing patient outcomes. In at least one embodiment, the data extraction process involves retrieving these specific data elements from the information data flows.

The ingestion module 302 can ingest information data flows without interfering with the normal flow of information data.

In at least one embodiment, the ingestion module 302 can process the information data flows using a variety of techniques such as, but not limited to, data transformation, data conversion and/or language detection. For example, data transformation can involve pre-processing data within the information data flows to remove irrelevant characters, to handle special characters and/or normalize text data. Data transformation can be implemented using natural language processing (NLP) libraries. Data conversion can involve converting data from one format to another. For example, data from a JSON file format may be converted to a CSV format. Language detection can involve Identifying the language of text data if the language is not specified, by parsing the text.

In at least one embodiment, the process of ingesting information data flows may include capturing metadata linked to the ingested data. This metadata can encompass various details, such as timestamps related to data acquisition, source information, and additional tags or labels. The captured metadata serves to track the origin of the data and provide context for its use.

In at least one embodiment, the process of ingesting information data flows may involve applying filters to manage the flow of data effectively. These filters operate by selectively permitting or restricting certain data elements based on predefined criteria, allowing for a more streamlined and efficient data processing experience.

By implementing these filters, the data management and analysis system 120 can ensure that only high-quality and relevant data is transmitted, which can be important to maintaining the integrity and reliability of the information being handled. Additionally, the filters can help ensure that the data delivered to external systems or end-users complies with specific data standards and business rules. This may involve checking for consistency, accuracy, and completeness of the data.

Furthermore, the use of filters can enhance the overall efficiency of the data ingestion process by reducing the volume of unnecessary or low-quality data, thereby optimizing system performance and ensuring that downstream applications receive only the most pertinent information for analysis or reporting. This capability is particularly beneficial in scenarios where large datasets are involved, as it allows organizations to focus on actionable insights and data-driven decision-making.

In at least one embodiment, the ingestion module 302 is configured to identify contextual factors present within the information data flows and to retrieve relevant data integration policies associated with these factors. For instance, if the information data flow pertains to an individual, the ingestion module 302 can identify demographic data related to that person. Additionally, if the context involves the size of the data, the ingestion module 302 can recognize volumetric data associated with the flow.

Once the contextual factors are identified, the ingestion module 302 label the contextual factors accordingly, allowing the data management and analysis system 120 to categorize different types of data based on their specific contextual attributes. This enables the separation of data into distinct groups for more effective parsing and analysis. For example, demographic data can be classified as one group, while volumetric data can be designated as another, facilitating targeted processing.

The identification of contextual factors may be informed by various types of information gathered from the data flows, including metadata. This metadata can provide insights into the nature and characteristics of the data, such as its source, acquisition date, and relevance to particular analysis frameworks. By leveraging these contextual factors and associated data integration policies, the ingestion module 302 can optimize the handling of diverse data types, ensuring that each group is processed with the appropriate methods and standards in mind. This capability enhances the overall efficiency and effectiveness of data analysis, leading to improved decision-making and insights based on the contextual relevance of the data.

In at least one embodiment, the information data flows can be logged and monitored using specialized logging and monitoring tools designed to track the health and performance of the ingestion module 302. This can involve implementing a systematic record-keeping process that captures and documents all actions and events related to the data intake process, from source to destination.

This systematic approach can provide a comprehensive chronological account of various data handling activities, including data extraction, transformation, validation, and loading. By maintaining an audit trail, the data management and analysis system 120 can enhance data integrity and security and ensure that any anomalies or discrepancies can be quickly identified and addressed.

The logging process can offer insights into compliance, as it can provide transparency regarding who accessed the data, the specific times of access, and any modifications or errors that may have occurred during processing. By tracking these metrics, organizations can ensure adherence to regulatory standards and best practices, ultimately fostering trust in the data management and analysis system 120.

Additionally, the logging and monitoring tools can facilitate proactive performance management by identifying potential bottlenecks or inefficiencies in the ingestion process. This allows for timely interventions and optimizations, ensuring that the ingestion module operates at peak efficiency and continues to meet the evolving needs of data analysis and reporting. Overall, the integration of logging and monitoring tools can strengthen the reliability and accountability of the data ingestion framework.

The parsing module 304 is configured to receive the ingested information data flows from the ingestion module 302 and to parse these data flows effectively. As noted, the ingested information may often be unstructured. The parsing module 304 can be configured to organize this unstructured data into a structured format.

Structuring unstructured information data flows can involve several processes, including processing the data flows to identify data element descriptors. Once these descriptors are determined, they can be transformed into parsed labels, enabling the data to be categorized and utilized effectively in subsequent analyses. This transformation can enhance the usability of the data, allowing for more efficient data management and integration within the overall system.

Processing the information data flows can include a variety of techniques aimed at transforming and enhancing the data for better usability. Processing the information data flows can involve data tokenization, which involves breaking down the information into manageable units or tokens for easier analysis. The tokens can involve various levels of granularity, such as individual words, sentences, or phrases. The parsing module 304 can then assign labels to the tokens, providing context to aid in understanding their meaning and significance within the data set.

Tokenization can facilitate analysis and processing of the data. For instance, when working with individual words, tokenization can allow for more straightforward operations like counting word frequency, identifying keywords, or performing sentiment analysis.

When sentences are used as tokens, the context of meaning can be preserved more effectively, which is particularly useful for applications such as natural language processing (NLP) and machine learning, where understanding the relationships between sentences can be important for generating coherent responses or insights.

Moreover, tokenization can also involve handling special cases, such as punctuation marks, numbers, and special characters, ensuring that they are treated appropriately within the context of the analysis. Additionally, tokenization may support the inclusion of multi-word expressions or phrases, which can convey specific meanings that individual words may not capture alone.

Token tagging can involve assigning each token to a specific category or group based on its characteristics. For instance, tokens that represent individual words can be tagged according to their grammatical classifications, such as nouns, verbs, adjectives, adverbs, and so on.

By categorizing tokens in this way, the data management and analysis system 120 can gain a deeper understanding of the grammatical structure of the text. Recognizing the roles that different words play in a sentence can allow the data management and analysis system 120 to analyze the text more effectively, improving its comprehension of the underlying meaning and context.

Token tagging can also facilitate advanced text processing tasks such as syntactic parsing, which examines the sentence structure to determine how words relate to one another. This is particularly valuable in applications like natural language processing (NLP), where accurate understanding of context and intent is crucial for generating coherent and relevant responses.

Additionally, tagged tokens can support various language processing tasks, including sentiment analysis, information extraction, and text classification. For example, knowing whether a token is a noun or a verb can enhance the data management and analysis system's 120 ability to identify key themes or subjects in a text, as well as discern the action or sentiment associated with those themes.

Moreover, token tagging can be extended to include additional layers of information, such as named entity recognition, which identifies proper nouns and classifies them as people, organizations, or locations. This enriched tagging provides a more comprehensive view of the data, enabling more sophisticated analyses and insight.

In at least one embodiment, the parsing process can involve dependency and relationship analysis, which examines the connections between different data elements, helping to reveal how they interact with and influence one another. Grammar and syntax analysis can be examined, as they ensure that the data adheres to the expected linguistic structures, thereby improving its quality and interpretability.

Dependency and relationship analysis can involve examining the connections and dependencies between tokens within the text. This process can be used for deciphering how different words and phrases interact with one another to form coherent sentences. For instance, the parsing module 304 may scrutinize sentences to uncover the grammatical relationships between words, identifying which words function as subjects, objects, or modifiers.

By parsing these dependencies, data management and analysis system 120 can gain insights into the syntactic structure of the text data, allowing it to comprehend the individual meanings of the words and how they work together to convey complete thoughts. This understanding can be important for applications in natural language processing (NLP), where grasping the context and meaning of a sentence can impact tasks such as sentiment analysis, machine translation, and text summarization.

Additionally, dependency analysis can help the data management and analysis system 120 identify more complex relationships, such as nested phrases or clauses, and how they relate to one another within a sentence. For example, in the sentence “The dog that chased the cat ran away,” the data management and analysis system 120 can determine that “the dog” is the subject while “that chased the cat” serves as a descriptive clause that modifies “the dog.”

Furthermore, this analysis can be enhanced by integrating semantic understanding, which involves recognizing the meanings behind the relationships. For instance, distinguishing between causative relationships (e.g., “The rain caused the flood”) and associative relationships (e.g., “The doctor treated the patient”) enables deeper comprehension of the text.

Grammar and syntax analysis can involve examining the grammatical and syntactic aspects of text to ensure proper structure and coherence. For instance, the parsing module 304 may employ natural language processing (NLP) techniques and libraries such as spaCy or NLTK, along with other methodologies, to perform this analysis on text data.

By leveraging these advanced NLP tools, the data management and analysis system 120 can assess the grammatical correctness of sentences, identify parts of speech, and evaluate sentence structure. This thorough analysis can enable the data management and analysis system 120 to comprehend the semantic structure of the data, which refers to the meaning conveyed by the arrangement of words and phrases.

Understanding the grammar and syntax of the text can allow the data management and analysis system 120 to parse sentences accurately, facilitating the extraction of meaningful information and insights. For example, by analyzing the syntactic structure, the data management and analysis system 120 can determine subject-verb-object relationships, identify modifiers, and recognize complex sentence constructions.

Moreover, grammar and syntax analysis can aid in detecting errors or inconsistencies within the text, such as subject-verb agreement issues or misplaced modifiers, thereby enhancing the overall quality and reliability of the data. By correcting these errors, the data management and analysis system 120 can produce more coherent and contextually relevant outputs.

Additionally, grammar and syntax analysis can contribute to various NLP applications, including machine translation, where maintaining grammatical accuracy is essential for producing intelligible translations, and sentiment analysis, where the sentiment conveyed can be influenced by syntactic structures.

In at least one embodiment, the parsing module 304 is configured for error detection. Error detection can involve identifying inconsistencies or inaccuracies within the data. Once errors are detected, the parsing module 304 can perform error correction to rectify any identified issues and error resolution processes to ensure that data integrity is maintained throughout.

In at least one embodiment, the parsing module can profile data. Data profiling involves a comprehensive examination of the data to assess its quality, structure, and content characteristics. Data profiling can help identify patterns and anomalies that may require attention. Additionally, the identification of missing values can be used to assess of the data set and allow for appropriate handling strategies to be applied, whether through imputation or exclusion.

Error detection can involve identifying various types of errors within data sets, ensuring the accuracy and integrity of the information being processed. Error detection can involve identifying typographical errors, including mistakes in text data, such as misspellings, incorrect word usage, or grammatical errors; detecting missing values which can occur when data is not collected or recorded correctly, leading to gaps in the dataset; identifying inconsistent formatting, including variations in how data is represented or structured, such as different date formats (e.g., MM/DD/YYYY vs. DD/MM/YYYY) or discrepancies in capitalization and abbreviation usage; identifying contradictory information (e.g., if one record indicates a patient is male while another entry states female); and/or identifying semantic conflicts arising from varying interpretations of terms or codes, which can lead to inconsistencies in data representation.

Error detecting can reduce misunderstandings or misinterpretations of data, help identifying gaps such as missing values, and standardize the data.

Effective error detection can improve the quality of the data and enhance the reliability of subsequent analyses and insights drawn from the information. By implementing robust error detection mechanisms, organizations can ensure that their data management systems maintain high standards of accuracy and usability. This leads to better decision-making processes and more reliable outcomes based on the analyzed data.

The parsing module 304 can be configured to resolve the errors detected. Error resolution can involve implementing specific strategies to address and resolve detected errors within a dataset. The chosen error resolution strategies can vary based on the type of error identified or the specific implementation of the data management and analysis system 120. Example error resolution techniques include: fuzzy matching techniques which can be employed to suggest corrections for erroneous values by comparing them to similar entries in the dataset. For instance, if a name is misspelled, fuzzy matching can identify the closest correct alternatives, providing recommendations for accurate replacements; data normalization and standardization, which can be used to correct inconsistent formatting across the dataset. For example, data normalization may involve converting all date formats to a standard format (e.g., YYYY-MM-DD) or ensuring consistent capitalization of names and categories. By standardizing the data, the data management and analysis system 120 can reduce ambiguity and enhance the overall quality and usability of the dataset; rule-based corrections which can involve implementing predefined rules that dictate how to address specific types of errors. For instance, if a numerical value falls outside a defined range, a rule can automatically flag it for review or suggest a correction based on historical data trends; manual review processes where automated strategies are insufficient or where errors are complex, involving human intervention may be necessary. Analysts can review flagged entries, ensuring that resolutions are contextually appropriate and accurate; feedback loops which can involve establishing mechanisms for ongoing learning from resolved errors. By analyzing patterns in past errors and corrections, the data management and analysis system 120 can improve its ability to detect and address similar issues more effectively over time.

Example imputation techniques include statistical methods including mean, median, or mode imputation used to replace missing values based on the distribution of the available data; regression-based imputation involving using regression models to predict missing values based on the relationships between different variables in the dataset; K-Nearest Neighbors (K-NN) imputation involving identifying the K closest records (neighbors) based on a defined distance metric and uses their values to estimate the missing data point; interpolation and extrapolation techniques involving estimating missing values based on the trends observed in neighboring data points. Interpolation fills in missing values within the range of existing data, while extrapolation extends this estimation beyond the available data points; semantic imputation, involving using contextual understanding of the data to infer missing values based on the meaning of related terms or entries; N-Gram Models, involving analyzing sequences of words or data points to predict missing values based on patterns observed in the dataset; Bayesian models, such as Latent Dirichlet Allocation (LDA) to estimate missing values, incorporating uncertainty in the imputation process; and fuzzy pattern matching, involving identifying missing values by comparing the existing data to patterns in the dataset, allowing for flexibility in matching similar entries.

In at least one embodiment, the information data flow ingested by the parsing module 304 incorporates labeled contextual factors that have been assigned by the ingestion module 302. These labeled contextual factors can provide insights into the nature and significance of the data, enabling the parsing module 304 to process the information more effectively. By having this contextual information readily available, the parsing module 304 can enhance its analysis and ensure that the data is interpreted in accordance with its specific characteristics and relevance, leading to improved overall data management and insights.

Determining data element descriptors can involve generating metadata or labels that convey essential information about the characteristics, structure, and context of the data. These descriptors can serve as informative tags that help users comprehend the content and meaning of the associated ingested data, thereby enhancing the overall management, searchability, and analysis of the data.

By creating these data element descriptors, the data management and analysis system 120 can facilitate effective data governance by providing clarity and consistency in how data is represented. This can be important in environments where data is sourced from multiple origins, as it allows for a unified understanding of the data elements regardless of their initial source. For example, descriptors can indicate the type of data (e.g., numerical, categorical, textual), the intended use or application of the data, and any relevant relationships to other data elements.

Furthermore, standardized descriptors can enhance interoperability among different systems and applications, making it easier to integrate and analyze data across various platforms. They also streamline data retrieval processes, allowing users to quickly locate specific data sets based on their descriptors. By ensuring that data elements are described uniformly, organizations can maintain a higher level of data integrity and coherence, which is crucial for informed decision-making and effective data-driven strategies.

In at least one embodiment, the parsing module 304 is configured to recognize and apply distinct descriptors to a single data element, irrespective of the order in which these descriptors were applied. For instance, if a descriptor is assigned to a data element before a specific event occurs, and then an additional or replacement descriptor is added after the event, the parsing module 304 can effectively identify both descriptors and differentiate the contexts in which they were applied.

This functionality can allow the data management and analysis system 120 to maintain a clear understanding of how the meaning and characteristics of the data element may evolve over time. By recognizing the historical and contextual significance of each descriptor, the parsing module 304 can ensure that subsequent analyses can accurately reflect the state of the data element at different points in time.

Moreover, this capability can enhance the data management and analysis system's 120 ability to provide context-aware insights, enabling users to interpret the data element more effectively based on its descriptors. For example, the data management and analysis system 120 can differentiate between the original context (prior to the event) and any modifications made later, allowing for more nuanced analysis and understanding of how the data may be impacted by various events or changes.

Transforming data element descriptors into parsed labels can involve analyzing and extracting specific information from these descriptors, which can facilitate the identification of various attributes, including data type, format, and relevant metadata. This transformation process can enhance the clarity and usability of the data.

By recognizing these distinct aspects, data parsing can allow for efficient processing and organization of the underlying data. For example, knowing the data type —whether it is numerical, categorical, or textual—can enable the data management and analysis system 120 to apply appropriate methods for analysis. Similarly, understanding the format (e.g., JSON, CSV) ensures that the data is correctly interpreted and handled.

Additionally, recognizing relevant metadata associated with the data elements can provide context that enhances the analytical capabilities of the data management and analysis system 120. This metadata may include information about the source of the data, its intended use, and any relationships to other data elements, all of which are critical for informed decision-making.

Transforming data element descriptors into parsed labels can streamline the utilization of data for various analytical and operational purposes and can enable more sophisticated data management practices, improving the overall efficiency of data workflows and facilitating deeper insights into the data's significance and implications. By organizing data in this structured manner, users can more easily access, analyze, and derive value from the information.

In at least one embodiment, the parsing module 304 can be utilized to map data element descriptors to parsed labels based on predefined mappings that are stored in memory. For instance, the module may map data element descriptors to specific names for pharmaceutical compounds, utilizing predefined mappings that are provided by an index file stored in the system's memory. This enables consistent and standardized representation of data across various datasets.

Alternatively, the parsing module 304 can perform mapping based on keywords and synonyms. For example, if a data element describes a symptom, such as “exhausted,” “tired,” or “sleepy,” the module can identify these terms as synonyms and map them to a standardized label for that symptom. By incorporating a set of common synonyms, the parsing module enhances the clarity and uniformity of the data, allowing for more effective analysis and reporting.

Through this labeling process, the parsing module 304 can generate modified information data flows that are enriched with standardized labels. This structured approach can facilitate easier data management but and improve the accuracy and relevance of subsequent analyses. By ensuring that similar concepts are consistently labeled, organizations can derive more meaningful insights from the data, enabling better decision-making and operational efficiency.

In at least one embodiment, the parsing module 304 is configured to assign a sensitivity level and/or various attributes to the data elements. For example, the parsing module 304 may associate each parsed label with specific factors that enhance the understanding and management of the data. These factors can include, but are not limited to: a sensitivity level indicating the degree of confidentiality or sensitivity associated with the data element, guiding how the information should be handled and protected. For instance, personal health information may be assigned a higher sensitivity level compared to general demographic data; policy violation severity, which can involve assessing the potential impact of a policy violation related to the data element. By categorizing the severity of possible violations, organizations can prioritize responses and interventions more effectively; a likelihood of occurrence evaluating how probable it is for a particular event or condition related to the data element to occur. Understanding this likelihood aids in risk assessment and resource allocation for preventive measures; a likelihood of detection gauging the chances of identifying issues or anomalies within the data. A higher likelihood of detection can lead to more proactive monitoring and management strategies.

In at least one embodiment, the parsing module 304 is configured to rank the severity of errors within the information data flows by assessing how significantly these errors disrupt the intended meaning and context of the data. Errors that severely undermine the semantics of the data, introduce ambiguity, or compromise its integrity in relation to its specific context are typically classified as severe.

The evaluation of error severity can also consider the impact on data relevance, the accuracy of insights derived from the data, and adherence to legal and regulatory requirements. Errors that are pervasive or have the potential to lead to incorrect conclusions can generally receive higher severity rankings. This prioritization allows for a more effective allocation of resources and efforts towards error resolution.

By incorporating semantic analysis, the parsing module 304 can help to focus on preserving data quality and ensuring meaningful interpretation of the data. This process can aid in identifying which errors require immediate attention and which can be addressed later, thus streamlining the overall data management workflow.

By closely monitoring these information data flows, the data management and analysis system 120 can promptly address any emerging issues, optimize performance, and maintain data consistency throughout the ingestion process. This proactive approach can enhance data integrity and foster trust in the insights generated from the data, ensuring that organizations can make informed decisions based on accurate and reliable information.

The semantic context and analysis module 306 can be configured to receive the modified information data flow from the parsing module 304. This modified data flow may include parsed labels that provide identifiers for the data elements, as well as various factors associated with these labels that enrich the understanding of the data's context and significance.

In a health-focused application, this the semantic context and analysis module 306 can analyze health-related data. For example, the parsed labels may correspond to specific medical conditions, treatments, or patient demographics, while the associated factors might include sensitivity levels indicating the confidentiality of patient information, severity rankings for potential errors in clinical data, or contextual attributes reflecting the relevance of the information to patient care.

By processing this enhanced data flow, the semantic context and analysis module 306 can derive meaningful insights that are essential for effective healthcare decision-making and enable healthcare professionals to better understand patient records, clinical trial data, or population health statistics, thus supporting improved patient outcomes.

Additionally, this semantic context and analysis module 306 can facilitate the identification of trends and patterns in health data, such as the prevalence of certain conditions within a population or the effectiveness of specific treatments. By incorporating semantic analysis, the data management and analysis system 120 can also enhance the accuracy of predictive modeling and risk assessment, allowing healthcare providers to allocate resources more effectively and tailor interventions to meet the unique needs of their patient populations.

The semantic context and analysis module 306 can enhance the overall usability of the information data flow, ensuring that healthcare organizations can derive valuable insights from their data while maintaining high standards of accuracy, privacy, and compliance with healthcare regulations. This capability is essential for fostering a data-driven approach to improving health outcomes and advancing public health initiatives.

In at least one embodiment, the semantic context and analysis module 306 is configured to evaluate the labeled information data flow thoroughly. This evaluation process involves determining the meaning and context of the modified information data flow, which is crucial for making informed decisions in a healthcare setting.

By analyzing the labeled data flow, the semantic context and analysis module 306 can extract critical insights regarding patient health conditions, treatment efficacy, and population health trends. For instance, the semantic context and analysis module 306 may assess how specific medical terms or diagnoses are represented in the data, allowing healthcare professionals to gain a clearer understanding of patient records and clinical documentation.

The evaluation can also encompass contextual factors such as the patient's demographic information, medical history, and relevant social determinants of health. By integrating these elements, the semantic context and analysis module 306 provides a comprehensive view of each data entry, facilitating better interpretations of patient needs and outcomes.

Understanding the meaning and context of the data allows for the identification of potential risks or anomalies in patient records. For example, if the data flow indicates a sudden increase in reported symptoms for a specific condition within a certain population, this could signal an emerging health concern that requires immediate attention from public health officials or healthcare providers.

The semantic analysis can enhance clinical decision support systems by providing relevant recommendations based on the evaluated data. For instance, it could suggest preventive measures or targeted interventions tailored to specific patient groups, thereby improving health outcomes.

The semantic context and analysis module 306 can help transform raw health data into meaningful insights, ensuring that healthcare providers can deliver high-quality care while also adhering to regulatory standards and maintaining patient privacy. This capability ultimately supports a more effective and proactive approach to healthcare management and decision-making.

In at least one embodiment, determining the meaning and context of the modified information flow involves identifying patterns, sentiments, and relationships within the data. The semantic context and analysis module 306 employs various advanced analytical techniques to extract valuable insights that are particularly relevant in the healthcare domain.

For instance, the semantic context and analysis module 306 can perform named entity resolution, which helps identify and categorize specific medical entities such as diseases, medications, and treatment protocols within the data. By disambiguating these entities, the semantic context and analysis module 306 can ensure that similar terms or concepts are accurately recognized, enhancing the clarity and reliability of the information.

The semantic context and analysis module 306 can be configured to employ various tools and software libraries to facilitate the identification and classification of these entities. For example, libraries such as spaCy and Stanford NER are commonly used for their robust capabilities in recognizing and categorizing entities within large volumes of text. These tools employ machine learning algorithms and natural language processing techniques to enhance the accuracy of entity recognition.

The types of identified entities can vary depending on the nature of the information data flows. In a healthcare setting, the identified entities can include any of the following: patient names identifying individuals involved in the medical records, ensuring that their information is correctly attributed and managed; healthcare organizations recognizing hospitals, clinics, and research institutions mentioned in the text, which can be important for understanding partnerships, referrals, and patient care pathways; and medical terms classifying specific medical conditions, treatments, and medications, enabling better tracking of health-related discussions and data.

By identifying these entities, healthcare providers can enhance data management and improve decision-making processes. For example, named entity resolution can facilitate patient matching in electronic health records (EHRs), improve the accuracy of health analytics, and aid in the extraction of insights from unstructured data sources.

Entity information can also support interoperability among healthcare systems and ensure that relevant data is easily retrievable and comprehensible, ultimately contributing to more effective patient care and management.

Entity disambiguation involves clarifying entities that may be ambiguous or difficult to classify accurately through standard named entity resolution techniques. In healthcare, many terms can refer to multiple concepts or entities, potentially leading to misunderstandings or errors in data interpretation.

The semantic context and analysis module 306 can be configured to analyze knowledge gained from the modified information data flow provided by the parsing module 304 to enhance the disambiguation process. By analyzing the surrounding context and relationships between entities, the module can differentiate between similar or identical terms based on their specific applications or meanings in a healthcare context.

For example, consider the term “Apple.” In a medical context, it could refer to either the fruit that may be mentioned in dietary recommendations or Apple Inc., which could be referenced in discussions about health technology products. Through entity disambiguation, the module determines the appropriate context for the term, ensuring accurate representation and understanding.

To facilitate this process, the semantic context and analysis module 306 can leverage various tools and software libraries, such as DBpedia, which provides a structured knowledge base that connects entities to their definitions, classifications, and relationships. By tapping into this rich resource, the semantic context and analysis module 306 can effectively resolve ambiguities and enhance the accuracy of entity classification.

In addition to knowledge bases like DBpedia, the semantic context and analysis module 306 may incorporate machine learning techniques and natural language processing algorithms to further refine its ability to disambiguate entities based on contextual clues, co-occurrences, and historical data patterns, allowing for more precise identification of entities, ultimately improving the quality and reliability of health data analysis.

By ensuring that entities are accurately disambiguated, healthcare organizations can minimize the risk of errors in clinical documentation, research findings, and data analysis. This capability enhances decision-making processes and supports improved patient care by providing clear, unambiguous information that healthcare professionals can trust. Overall, entity disambiguation can help transform complex, unstructured health data into actionable insights that drive better health outcomes.

The semantic context and analysis module 306 can be configured to perform sentiment analysis and assess the emotional tone or attitude expressed within patient feedback, clinical notes, or social media discussions related to health. This analysis can help healthcare providers gauge patient satisfaction, identify concerns, and understand public perception regarding specific treatments or healthcare services.

In at least one embodiment, the semantic context and analysis module 306 can engage in topic modeling, which identifies prevalent themes or topics within the modified information flow. For example, semantic context and analysis module 306 can reveal trends in patient symptoms, emerging health issues in specific populations, or the effectiveness of particular interventions over time. By understanding these topics, healthcare organizations can better allocate resources and address pressing health concerns.

Relationship extraction can enhance the analysis by uncovering the connections between various entities and attributes in the data. For instance, relationship extraction can identify relationships between patients'demographics and their health outcomes, helping to reveal potential disparities or risk factors that need to be addressed.

The semantic context and analysis module 306 can be configured to use ontologies and graph creation to structure health-related data. By building a semantic network that represents the relationships between different medical concepts, the semantic context and analysis module 306 can facilitate more intuitive data navigation and retrieval. This structure allows for a richer understanding of how various health conditions are interconnected, aiding in comprehensive patient care and research.

The semantic context and analysis module 306 can be configured to synthesize the insights obtained to provide a holistic view of the modified information flow, enabling healthcare providers to make more informed decisions based on the collective understanding of patient needs, treatment effectiveness, and broader health trends.

Sentiment analysis can involve identifying and interpreting the tone or sentiment conveyed in text. In a healthcare context, this analysis can be particularly valuable, as it allows healthcare providers, researchers, and organizations to gauge patient emotions, perceptions, and overall sentiment regarding treatments, healthcare services, and health-related topics.

The semantic context and analysis module 306 can be configured to use various models for conducting sentiment analysis, approaches to those of ordinary skill in the art, based on recurrent neural networks (RNNs) and transformer models. These advanced models leverage deep learning techniques to analyze text, enabling the data management and analysis system 120 to capture nuanced sentiments and contextual meanings that may not be immediately apparent.

During the sentiment analysis process, the semantic context and analysis module 306 can assign sentiment scores to different segments of text, categorizing them as positive, negative, or neutral. For instance, patient feedback from surveys or online reviews may reveal a wealth of information about their experiences with healthcare providers, treatments, or facilities. A positive sentiment score might indicate satisfaction with care received, while a negative score could highlight concerns or dissatisfaction that may require further investigation.

Additionally, sentiment analysis can include a more detailed understanding of patient attitudes. For example, a healthcare provider might analyze social media posts or online forums to assess public sentiment regarding a new vaccine. By quantifying the proportion of positive, negative, and neutral sentiments, the organization can gauge community reactions, identify potential areas of resistance, and tailor communication strategies accordingly.

Moreover, sentiment analysis can enhance clinical decision-making by enabling healthcare professionals to recognize trends in patient emotions and concerns. Understanding patient sentiment can help providers tailor their communication and interventions to better address the emotional and psychological aspects of patient care, leading to improved health outcomes.

Topic modeling can be used to uncover latent topics or themes within text data. In the healthcare domain, this process is particularly valuable for managing and interpreting large volumes of clinical documentation, patient feedback, research articles, and other textual sources.

The semantic context and analysis module 306 can employ various topic modeling techniques, including well-established methods such as Latent Dirichlet Allocation (LDA) and Non-Negative Matrix Factorization (NMF). LDA is a probabilistic model that assumes each document is generated from a mixture of topics, while NMF decomposes the text data into non-negative factors that represent underlying topics. Both approaches can enable the semantic context and analysis module 306 to identify common themes, trends, and patterns that may not be immediately visible through traditional text analysis methods.

By performing topic modeling, the data management and analysis system 120 can effectively categorize and organize text based on these underlying topics, allowing for a more structured approach to data analysis. For instance, in a clinical setting, topic modeling can reveal prevalent themes in patient notes, identifying common symptoms or concerns that may warrant further investigation. Topic modeling can also be used to analyze patient feedback from surveys or online platforms, enabling healthcare organizations to understand the key issues affecting patient satisfaction and care quality.

In addition to improving data organization, topic modeling can support more targeted research initiatives. For example, by identifying topics related to emerging health conditions or treatments within published literature, researchers can quickly pinpoint areas that require further exploration or intervention. This capability is particularly beneficial in rapidly evolving fields such as public health, where timely insights are crucial for addressing pressing health concerns.

Furthermore, by recognizing the themes and topics that resonate most with patients and healthcare providers, organizations can tailor their communications, educational materials, and outreach efforts to address specific interests and concerns. This targeted approach enhances engagement and supports better health literacy among patients.

Relationship extraction can involve identifying and extracting relationships or connections between entities mentioned within text data. This analytical technique can enable the discovery of intricate relationships that can inform clinical decisions, research, and patient care.

For instance, within a clinical study report, the semantic context and analysis module 306 may identify relationships such as a specific patient being treated with a particular medication, or a healthcare provider collaborating with a specific research institution. By extracting these connections, the data management and analysis system 120 can generate insights into how different entities interact within the healthcare landscape.

Additionally, relationship extraction can be applied to electronic health records (EHRs), where the module might identify relationships between patients and their medical histories, treatments, or outcomes. For example, relationship extraction can recognize that a patient with diabetes is receiving treatment from an endocrinologist, which is critical information for coordinating care and ensuring effective management of the patient's health condition.

Furthermore, relationship extraction can help in understand broader health trends and associations. For example, by analyzing public health data or research publications, the semantic context and analysis module 306 can uncover relationships between specific demographics and the prevalence of certain health conditions. This can help identify at-risk populations, guiding targeted interventions and resource allocation.

In the context of patient feedback and satisfaction surveys, relationship extraction can also reveal connections between patients'experiences and specific healthcare services or practices. For instance, the semantic context and analysis module 306 may identify a relationship between positive patient outcomes and particular treatment protocols or healthcare providers, helping to enhance the quality of care offered.

Ontology or graph creation can involve utilizing ontologies or knowledge graphs, such as the Resource Description Framework (RDF) and Web Ontology Language (OWL), to develop structured representations of the modified information data flows. In the context of healthcare, these graphs serve as tools for organizing and visualizing complex relationships between various medical concepts, patient data, treatments, and outcomes.

By creating these ontologies or graphs, the semantic context and analysis module 306 can illustrate the connections among different entities and concepts, enhancing the understanding of how they interact within the healthcare ecosystem. For example, a knowledge graph might represent relationships between diseases, symptoms, treatment protocols, and patient demographics, providing a comprehensive view of patient care pathways and clinical decision-making processes.

The structured nature of these graphs can enable the data management and analysis system 120 to contextualize the modified information data flows more effectively. This contextualization can help ensure that healthcare professionals can interpret the data in relation to relevant medical knowledge, improving the accuracy and relevance of analyses conducted on patient records, clinical trial data, or public health information.

Moreover, the creation of ontologies facilitates semantic analysis of the modified information data flows, allowing the data management and analysis system 120 to apply meaning to the data beyond simple keyword matching. For instance, it can help differentiate between similar medical terms, clarify their implications based on context, and provide insights that guide clinical decisions. This capability is particularly valuable when analyzing unstructured data sources, such as clinical notes or patient feedback, where the nuances of language can significantly impact understanding.

Contextual analysis can involve interpreting the meaning of specific phrases, words, or paragraphs within text data based on their surrounding context. This approach recognizes that the meaning of language is often dependent on adjacent words or phrases, allowing for a more nuanced understanding of the content.

In a healthcare setting, the semantic context and analysis module 306 can utilize advanced contextual embedding techniques to enhance this analysis. Techniques such as Embeddings from Language Models (ELMo) and Bidirectional Encoder Representations from Transformers (BERT) are particularly effective for capturing context-aware representations of words and phrases.

For instance, in medical records or clinical notes, the term “diabetes” could take on different meanings depending on its context. When analyzing a sentence like “The patient with diabetes reported feeling fatigued,” the contextual analysis would consider surrounding terms to understand that the fatigue may be related to the patient's diabetes management or complications. Similarly, in a research paper, the phrase “insulin sensitivity” might be better understood when placed in the context of discussions about obesity or metabolic syndrome.

By applying these contextual embedding techniques, the semantic context and analysis module 306 can develop a more comprehensive representation of language that reflects its meaning in specific contexts and enable healthcare professionals to extract relevant insights from unstructured text data, improving clinical decision-making and patient care. For example, the semantic context and analysis module 306 can facilitate the identification of key symptoms, potential drug interactions, or patient sentiments in feedback forms.

Additionally, context-aware representations can enhance the accuracy of predictive analytics, such as identifying patients at risk for certain conditions based on their descriptions of symptoms. By understanding the context in which specific terms are used, healthcare organizations can improve their diagnostic capabilities and tailor interventions more effectively.

In at least one embodiment, the semantic context and analysis module 306 is configured to generate a comprehensive report based on the evaluation of the modified information data flow. This report can synthesize the insights derived from the contextual analysis, entity recognition, sentiment evaluation, and relationship extraction processes performed on the health-related text data.

The generated report can serve as a resource for healthcare professionals, presenting key findings and actionable insights in a clear and organized format. For example, the report might highlight prevalent health concerns identified through patient feedback, summarize the sentiment expressed in clinical notes, or detail the relationships between different symptoms and diagnoses. Such a report can be instrumental in informing clinical decisions, guiding treatment plans, and improving patient care strategies.

Once compiled, the report can be delivered to users through various interfaces, such as user device 104, which could include tablets, smartphones, or desktop computers. This accessibility ensures that healthcare providers have real-time access to critical information when they need it most, enhancing their ability to make informed decisions and respond effectively to patient needs.

Additionally, the report can be customized to cater to specific user roles or preferences. For instance, a physician may receive a report focused on clinical insights and treatment recommendations, while a healthcare administrator may be more interested in aggregated data on patient satisfaction and operational efficiency.

In at least one embodiment, the semantic context and analysis module 306 is configured to evaluate the labeled information data flows using a set of analytic rules. This evaluation occurs on a rule-by-rule basis, allowing for a systematic approach to identify and prioritize errors or omissions in data sets. Factors that influence this prioritization include data sensitivity, violation severity, likelihood of occurrence, and likelihood of detection, which may be previously determined by the parsing module 304.

For instance, in a healthcare setting, the semantic context and analysis module 306 may prioritize data related to patient safety, such as medication interactions or allergy information, which are deemed sensitive and critical. By applying these analytic rules, the semantic context and analysis module 306 can effectively process modified data information flows, examining how any alterations impact the underlying meaning, context, and relationships of the data.

As part of this analysis, the semantic context and analysis module 306 can evaluate modifications to determine whether they align with the original semantic meaning of the data. For example, if a data entry regarding a patient's diagnosis is altered, the semantic context and analysis module 306 can verify the new information is consistent with the patient's medical history and prior entries. This ensures that changes do not inadvertently misrepresent the patient's condition or treatment plan.

The analysis considers the implications of modifications on data relationships, dependencies, and overall relevance. It assesses whether changes introduce inconsistencies or contradictions that could lead to confusion or misinterpretation among healthcare providers. For example, if a patient's treatment history indicates a specific allergy, any modification that contradicts this information could be flagged as a significant error.

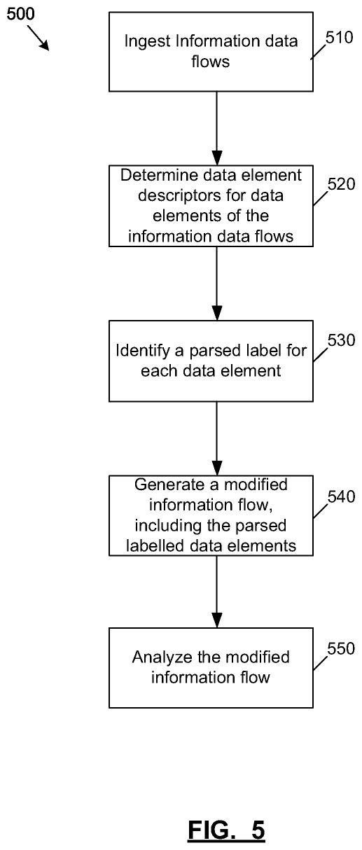

Referring to FIG. 5, shown therein is flowchart of a method 500 for managing and analyzing information data flows, in accordance with one embodiment. The method 500 can be implemented by the data management and analysis system 120. The method can involve data ingestion, parsing, and contextual analysis to interpret the meaning of phrases and terms based on their surrounding context. The method can also involve error detection and correction, identifying discrepancies and inconsistencies within the data, sentiment analysis and topic modeling to gauge patient satisfaction and identify prevalent themes in feedback or clinical discussions, and the generation of comprehensive reports that synthesize the findings from the analysis. These reports can be provided to healthcare professionals with actionable insights, enabling them to make informed decisions based on a thorough understanding of patient data and health trends.

At step 510, the ingestion module 302 of the system 120 ingests information data flows and produces ingested information data flows. Ingesting the information data flows can involve processing data as described with reference to FIG. 3. For example, the ingestion module 302 can retrieve data from various sources, including electronic health records (EHRs), clinical notes, patient surveys, laboratory results, and other health-related data repositories. As described, the information data flows can include data elements. The information data flows can originate from various sources of information, including electronic health records, clinical notes, and patient feedback. The information data flows can contain various data elements, which may include patient information such as demographic details, medical histories, treatment plans, and real-time health metrics collected from wearable devices. By processing these data elements, the ingestion module 302 can capture information and format the information for further analysis.

In at least one embodiment, the parsing module 302 is configured to clean the data to remove duplicates or irrelevant information, perform data normalization to standardize formats, and/or perform metadata tagging to enhance the understanding of each data element's context and relevance.

By effectively ingesting and processing these information data flows, the ingestion module 302 can enable healthcare providers to access a rich repository of data that can inform clinical decision-making, improve patient outcomes, and facilitate targeted interventions.

The parsing module 304 then receives the ingested information data flows. At step 520, the parsing module 304 determines data element descriptors for each of the data elements of the ingested information data flows. The parsing module 304 can process the ingested information data flows to identify key elements, label data, and apply semantic context to facilitate better understanding and organization of the information, as explained with reference to FIG. 3.

In at least one embodiment, the parsing module 304 examines the content for key elements and patterns. This may involve identifying various data types, such as text, numerical values, and categorical data, and organizing them in a coherent manner. In a healthcare context, this could include parsing clinical notes to extract patient symptoms, medication details, and treatment outcomes, as well as structuring demographic information and laboratory results.

In at least one embodiment, the parsing module 304 labels the identified data elements, applying appropriate descriptors that clarify their meaning and relevance. For instance, a parsed label may indicate whether a specific entry corresponds to a diagnosis, a treatment protocol, or a patient-reported outcome. This structured labeling is essential for ensuring that the subsequent analyses are accurate and contextually relevant.

In at least one embodiment, the parsing module 304 implement data validation checks to ensure the integrity of the information. By confirming that the data adheres to predefined standards and formats, the parsing module 304 can help minimize errors that could compromise the quality of health data analysis.

By converting raw data into a structured and labeled format, the parsing module 304 can enable healthcare organizations to effectively utilize the information for clinical decision-making, patient management, and operational improvements, ultimately leading to enhanced patient care and better health outcomes.

In at least one embodiment, the parsing module analyzes 304 each data element to create detailed descriptors that encapsulate essential information such as the data type (e.g., numerical, categorical, textual), format (e.g., date, percentage, string), and any relevant contextual metadata. For instance, in a healthcare setting, if the data element pertains to a patient's vital signs, the descriptor might include attributes like the measurement type (e.g., blood pressure, heart rate), the unit of measurement (e.g., mmHg for blood pressure), and the date and time of the reading.

By generating these descriptors, the parsing module 304 can enhance the usability of the data elements, allowing healthcare professionals to quickly grasp their meaning and context. This can be important in scenarios where clinicians need to interpret complex health data, such as interpreting a patient's history of symptoms or treatment responses.

The data element descriptors can facilitate better data management practices by ensuring that the information can be easily searched, categorized, and analyzed. For example, the data element descriptors can enable grouping of similar data elements together, making it easier to conduct analyses on patient populations, track health outcomes, and identify trends in treatment efficacy.

Well-defined data element descriptors can contribute to data integrity and compliance with healthcare regulations. Accurate descriptors can help ensure that sensitive patient information is handled appropriately and that data sharing across systems adheres to privacy standards.

At step 530, the parsing module 304 identifies a parsed label for each data element within the ingested information data flows.

The parsed label can be determined based on the previously established data element descriptor, which can outline the characteristics and context of the data element. For example, if the data element pertains to a patient's medical condition, the parsed label might specify the condition's name (e.g., “hypertension”) along with any relevant identifiers, such as ICD-10 codes, that facilitate standardization and interoperability across different healthcare systems.

By assigning these parsed labels, the parsing module 304 can enhance the clarity and usability of the data elements. In healthcare settings, this means that clinicians and researchers can quickly identify and interpret key pieces of information related to patient care, treatment plans, and health outcomes. For instance, having a clearly defined parsed label for a medication can help ensure that healthcare providers understand its proper usage, dosage, and potential side effects.

Additionally, parsed labels can facilitate data integration and analysis. When data elements are labeled consistently, it becomes easier to aggregate information from various sources, compare findings, and conduct comprehensive analyses. For example, parsed labels can enable healthcare organizations to track trends in disease prevalence, treatment efficacy, or patient satisfaction over time, leading to more informed decision-making and improved healthcare delivery.

Moreover, the use of parsed labels can enhance compliance with healthcare regulations and standards, such as HIPAA and FHIR. By ensuring that data is accurately labeled and categorized, healthcare providers can better manage sensitive patient information and maintain transparency in data sharing practices.

The identification of parsed labels can transform the data into meaningful, organized information that can be used for effective patient care, research, and operational efficiency in the healthcare system. This process not only supports enhanced data management but also contributes to better health outcomes by enabling healthcare professionals to make well-informed decisions based on accurate and easily accessible information.

At step 540, the parsing module 304 generates modified information data flows that incorporate the parsed labeled data elements. The modified information data flows can be further analyzed. By embedding the parsed labels within the data flows, the parsing module 304 can ensure that each data element is clearly defined and contextualized, enhancing its usability in subsequent processing stages.

The modified information data flows can contain details that allow healthcare professionals to interpret the data more effectively. For instance, a data flow that previously contained unstructured clinical notes can now present clearly labeled symptoms, treatments, and patient demographics, enabling easier identification of trends and patterns. This structured format is particularly valuable in healthcare, where the clarity of information can significantly impact clinical decision-making and patient outcomes.

After generating these modified information data flows, the parsing module 304 can transmit the modified information data flows to the semantic context and analysis module 306. The semantic context and analysis module 306 can use the structured data to conduct a variety of evaluations, including contextual analysis, relationship extraction, and sentiment analysis, among others.

The integration of parsed labeled data elements into the modified information data flows allows the semantic context and analysis module 306 to derive deeper insights from the data. For example, semantic context and analysis module 306 can assess how specific symptoms are related to certain conditions or treatments, analyze patient feedback to gauge satisfaction, and identify emerging health trends based on structured data from multiple sources.

At step 550, the semantic context and analysis module 306 analyzes the modified information data flows to extract insights that can inform healthcare decisions.

The analysis of the modified information data flows involves determining the meaning and context of the data. The semantic context and analysis module 306 can use various analytical techniques to identify significant patterns, sentiments, and relationships within the data, as described with reference to FIG. 3. For example, by examining patient records and feedback, the semantic context and analysis module 306 can uncover trends in patient symptoms or treatment responses, providing valuable information to healthcare providers about how different patient populations may be affected by specific interventions.

In at least one embodiment, the semantic context and analysis module 306 performs sentiment analysis to gauge patient perceptions and experiences related to their healthcare. Understanding the emotional tone expressed in patient feedback—whether it is positive, negative, or neutral—can help healthcare organizations identify areas for improvement in patient care and enhance overall satisfaction.

In at least one embodiment, the semantic context and analysis module 306 performs relationship extraction, which identifies connections between various data elements, such as the correlation between specific symptoms and their respective treatments or the relationships among different health conditions. Relationship extraction can help in managing complex cases where multiple conditions may coexist, as it enables healthcare providers to develop more tailored treatment plans that address the unique needs of each patient.

The semantic context and analysis module 306 may also leverage advanced techniques such as topic modeling to uncover underlying themes within the data. This can involve identifying common topics in patient feedback or research articles that relate to emerging health concerns, allowing organizations to respond proactively to public health trends or areas of patient interest.

The analysis of modified information flows can enable healthcare providers to make informed decisions, improve patient outcomes, and drive continuous enhancements in the quality of care delivered across the healthcare system.

In at least one embodiment, the semantic context and analysis module 306 produces a report that encapsulates the findings derived from its analysis of the modified information data flows. This report serves as a critical tool for healthcare professionals and administrators, providing a detailed overview of various aspects of the data, including detected errors, omissions, imputations, and corrections made during the data processing stages.

The report may include a section dedicated to identifying specific errors found within the data. These could range from typographical mistakes in patient records to inconsistencies in medication dosages or conflicting patient histories. Highlighting these errors can help ensure data integrity, as it allows healthcare providers to address potential issues that could impact patient safety and treatment outcomes.

The report can detail any omissions identified in the data. This may include missing values or incomplete records that could hinder comprehensive analysis. By pinpointing these gaps, the report can enable healthcare teams to take corrective actions, such as reaching out to patients for additional information or reviewing clinical documentation practices.

The report can outline the imputations performed to fill in missing values. For instance, if patient vital signs were incomplete, the report may summarize the statistical methods or machine learning algorithms used to estimate and fill these gaps. By documenting these imputation techniques, the report can enhance transparency and allow healthcare professionals to understand how decisions were made regarding the data.

The report may highlight any corrections that have been made during the data processing phase. This could include adjustments to erroneous data entries based on validated sources or updated clinical guidelines. By providing this level of detail, the report fosters confidence in the data being used for clinical decision-making.

The report generated by the semantic context and analysis module 306 can help ensure the quality and reliability of healthcare data. By offering insights into errors, omissions, imputations, and corrections, the report can support healthcare providers in their efforts to deliver safe and effective care, ultimately contributing to better patient outcomes and enhanced operational efficiency within healthcare organizations.

In at least one embodiment, the semantic context and analysis module 306 detects errors and omissions in the data by comparing the content and context of the ingested information against predefined patterns, rules, or reference data. This comparison can enable the semantic context and analysis module 306 to identify inconsistencies such as missing values, data outliers, or contradictory information that may signify potential errors or omissions in patient records or other health-related datasets.

For instance, in a clinical setting, the semantic context and analysis module 306 may flag a situation where a patient's medication list includes a dosage that is unusually high compared to established guidelines, indicating a possible error in data entry. Similarly, if a patient's allergy information is incomplete or conflicting with treatment records, this could alert healthcare providers to a critical oversight that needs immediate attention.

The semantic context and analysis module 306 can also help identify anomalies within data relationships and structures. By examining how different data elements interact with one another, the semantic context and analysis module 306 can detect unusual patterns that may suggest underlying issues. For example, if a patient's lab results indicate elevated blood glucose levels, but their records show no history of diabetes, this anomaly could prompt further investigation into potential data inaccuracies or gaps in the patient's medical history.

By facilitating early detection of such issues, the semantic context and analysis module 306 can help prevent complications that could arise from overlooked errors during the data ingestion process. This proactive approach is essential can help maintain the integrity of health data and ensure that healthcare providers have access to accurate and reliable information for decision-making.

The generated report summarizing these findings can be delivered to users through various means, such as user device 104, ensuring that healthcare professionals receive timely insights to address identified issues. Additionally, the report can be stored in memory within a data store such as the external data storage 108 or on a user device 104, allowing for easy retrieval and review. This capability not only enhances the workflow for healthcare providers but also contributes to ongoing quality improvement efforts by providing a historical record of identified errors and actions taken to rectify them.

The ability of the semantic context and analysis module 306 to detect and report on data inconsistencies is a component of effective data management in healthcare, leading to improved patient safety, better clinical outcomes, and enhanced operational efficiency within healthcare organizations.

While the applicant's teachings described herein are in conjunction with various embodiments for illustrative purposes, it is not intended that the applicant's teachings be limited to such embodiments. On the contrary, the applicant's teachings described and illustrated herein encompass various alternatives, modifications, and equivalents, without generally departing from the embodiments described herein. For example, while the teachings described and shown herein may comprise certain elements/components and steps, modifications may be made as is known to those skilled in the art. For example, selected features from one or more of the example embodiments described herein in accordance with the teachings herein may be combined to create alternative embodiments that are not explicitly described. All values and sub-ranges within disclosed ranges are also disclosed. The subject matter described herein intends to cover and embrace all suitable changes in technology.

1. A system for generating a health outcome prediction for a patient, the system comprising at least one processor configured to:
receive a health condition of interest;
receive a health dataset comprising at least patient data associated with the patient at least a portion of the health dataset being unstructured data, wherein the health dataset originates from a plurality of distinct data sources, and wherein the patient data comprises at least demographic information about the patient and clinical information;
process the unstructured data of the health dataset to obtain structured data;
extract from the processed health dataset, a plurality of features determined to be relevant to the health condition of interest;
determine a risk score for the patient for the health condition of interest using a trained health prediction engine, based on the plurality of features extracted, wherein the trained health prediction engine is configured to select one or more prediction models for determining the risk score based on composition of the health dataset; and
generate and display, on a graphical user interface, a graphical output based on the risk score.

2. The system of claim 1, wherein the health dataset includes one or more of genomic information, population data, lifestyle information, temporal information, environmental information, and sensor data.

3. The system of claims 1, wherein the at least one processor is configured to determine a recommendation based on the risk score and the health condition of interest.

4. The system of claim 3, wherein: (i) the recommendation is one of a treatment, a targeted therapy, a monitoring strategy or (ii) the recommendation is further based on environmental conditions and a type of health condition, and the recommendation is one of a resource allocation recommendation and an evacuation recommendation.

5. The system of claim 1, wherein: (a) the health dataset comprises real-time patient data from one of an emergency response dispatch system and emergency incident reports, (b) the graphical output is a visual alert, and/or (c) the risk score is associated with a timeline.

6. The system of claim 1, wherein a parsing module is configured to tokenize the unstructured data into a plurality of data tokens and assign corresponding tags to the plurality of data tokens according to grammatical classifications.

7. The system of claim 1, wherein the health prediction engine determines the risk score based on correlations between the plurality of relevant features and health outcomes.

8. The system of claim 1, wherein the unstructured data comprises data elements, and wherein processing the unstructured data comprises:
determining, via a parsing module, data element descriptors for each of the data elements of the unstructured data;
identifying, via the parsing module, a corresponding parsed label for each of the data elements, the parsed label identified based on the data element descriptor;
generating, via the parsing module, modified information data flows including the parsed labelled data elements; and
analyzing, via a semantic context and analysis module, the modified information data flows to determine a meaning and context of the modified information data flows.

9. The system of claim 8, wherein identifying the corresponding parsed label comprises one of: matching a given data element according to a predefined parsed label mapping, and determining a given parsed label based on one or more of keywords and synonyms of the given data element.

10. The system of claims 8, wherein analyzing the modified information data flows comprises comparing the modified information data flows with analytic rules to identify one or more of errors and omissions in the modified information data flows.

11. The system of claim 8, wherein: (I) the parsing module is configured to determine one or more of a sensitivity level, a violation severity, a likelihood of occurrence, and a likelihood of detection for each data element; (II) the semantic context and analysis module is further configured to generate a report including identified errors or omissions in the modified information data flows corresponding to the information data flows; and/or (III) determining the meaning and context of the modified information data flows comprises identifying patterns, sentiments and relationships within the modified information data flows.

12. A method for generating a health outcome prediction for a patient, the method comprising:
receiving a health condition of interest;
receiving a health dataset comprising at least patient data associated with the patient, at least a portion of the health dataset being unstructured data, wherein the health dataset originates from a plurality of distinct data sources, and wherein the patient data comprises at least demographic information about the patient and clinical information;
processing the unstructured data of the health dataset to obtain structured data;
extracting from the processed health dataset, a plurality of features determined to be relevant to the health condition of interest;
determining a risk score for the patient for the health condition of interest using a trained health prediction engine, based on the plurality of features extracted, wherein the trained health prediction engine is configured to select one or more prediction models for determining the risk score based on composition of the health dataset; and
generating and displaying, on a graphical user interface, a graphical output based on the risk score.

13. The method of claim 12, wherein the health dataset includes one or more of genomic information, population data, lifestyle information, temporal information, environmental information, and sensor data.

14. The method of claim 12, further comprising determining a recommendation based on the risk score and the health condition of interest.

15. The method of claim 14, wherein the recommendation is one of a treatment, a targeted therapy, a monitoring strategy or wherein the recommendation is further based on environmental conditions and a type of health condition, and the recommendation is one of a resource allocation recommendation and an evacuation recommendation.

16. The method of claim 12, wherein the risk score is associated with a timeline.

17. The method of claim 12, wherein the method comprises, via a parsing module, tokenizing the unstructured data into a plurality of data tokens and assigning corresponding tags to the plurality of data tokens according to grammatical classifications; and/or wherein the health prediction engine determines the risk score based on correlations between the plurality of relevant features and health outcomes.

18. The method of claim 12, wherein the unstructured data comprises data elements, and wherein processing the unstructured data comprises:
determining, via a parsing module, data element descriptors for each of the data elements of the unstructured data;
identifying, via the parsing module, a corresponding parsed label for each of the data elements, the parsed label identified based on the data element descriptor;
generating, via the parsing module, modified information data flows including the parsed labelled data elements; and
analyzing, via a semantic context and analysis module, the modified information data flows to determine a meaning and context of the modified information data flows.

19. The method of claim 18, wherein identifying the corresponding parsed label comprises one of: matching a given data element according to a predefined parsed label mapping, and determining a given parsed label based on one or more of keywords and synonyms of the given data element.

20. The method of claim 18, wherein analyzing the modified information data flows comprises comparing the modified information data flows with analytic rules to identify one or more of errors and omissions in the modified information data flows.

21. The method of claim 18, further comprising: (a) determining, by the parsing module, one or more of a sensitivity level, a violation severity, a likelihood of occurrence, and a likelihood of detection for each data element; (b) generating, by the semantic context and analysis module, a report including identified errors or omissions in the modified information data flows corresponding to the information data flows; and/or (c) determining the meaning and context of the modified information data flows comprises identifying patterns, sentiments and relationships within the modified information data flows.
